(19) United States
(12) Patent Application Publication
(10) Pub. No.: US 2015/0118221 A1
(43) Pub. Date: Apr. 30, 2015

(54) COMPOSITIONS AND METHODS FOR TREATING CARDIAC HYPERTROPHY

(71) Applicant: THE JOHNS HOPKINS UNIVERSITY, Baltimore, MD (US)

(72) Inventor: Subroto Chatterjee, Columbia, MD (US)

(73) Assignee: The John Hopkins University, Baltimore, MD (US)

(21) Appl. No.: 14/399,586

(22) PCT Filed: May 8, 2013
(86) PCT No.: PCT/US2013/040187

§ 371 Date: Nov. 7, 2014

Related U.S. Application Data
(60) Provisional application No. 61/643,932, filed on May 8, 2012.

(51) Int. Cl. A61K 31/5375; C12Q 1/48; G01N 33/68; A61K 31/445; G01N 33/573

(52) U.S. Cl. CPC A61K 31/5375; A61K 31/445; G01N 33/573; G01N 33/6893; C12Q 1/48; G01N 2800/324; G01N 2333/91102
USPC 424/130.1; 514/237.8; 514/315; 514/44 A; 514/1.1; 435/7.4; 435/15; 506/9

(57) ABSTRACT

The present invention relates to the field of cardiology. More specifically, the present invention provides methods and compositions useful for treating cardiac hypertrophy. In a specific embodiment, a method for treating or preventing cardiac hypertrophy in a patient comprises the step of administering a therapeutically effective amount of a glycolipid synthesis inhibitor.

### CROSS-REFERENCE TO RELATED APPLICATIONS

This application claims the benefit of U.S. Provisional Application No. 61/643,932, filed May 8, 2012; which is incorporated herein by reference in its entirety.

### STATEMENT OF GOVERNMENTAL INTEREST

This invention was made with U.S. government support under grant no. NIH PO1-HL-107153-01. The U.S. government has certain rights in the invention.

### FIELD OF THE INVENTION

The present invention relates to the field of cardiology. More specifically, the present invention provides methods and compositions useful for treating cardiac hypertrophy.

### BACKGROUND OF THE INVENTION

Cardiac hypertrophy is an adaptive response of the heart to virtually all forms of cardiovascular disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to dilated cardiomyopathy, heart failure, and sudden death. Despite the development and availability of many methods for diagnosis and treatment of cardiac conditions, the morbidity and mortality related to cardiac hypertrophy remains very high.

### SUMMARY OF THE INVENTION

The present invention is based, at least in part, on the discovery that glycolipid synthesis inhibitors can be used to treat or prevent cardiac hypertrophy and aortic thickening. The present invention is also directed to the use of glycosyltransferases as biomarkers in cardiac hypertrophy and aortic thickening. The present invention is also directed to the use of glycolipid synthesis inhibitors to treat or prevent atherosclerosis. Indeed, glycolipid synthesis inhibitors provide cardioprotective, anti-atherogenic and de-stiffening effects.

Cardiac hypertrophy, increased aortic intima-media thickening and increased blood pressure are the hallmarks of coronary artery disease. The present inventors have examined whether the inhibition of glycolipid synthesis can ameliorate heart disease using a transgenic mouse model of heart disease—the apoE−/− mice. Feeding a high fat and cholesterol diet to these apoE−/− mice markedly accelerates atherosclerosis and is accompanied by increased activity of glycolipid-glycosyltransferase blood pressure, aortic initma media thickening and cardiac dysfunction compared to mice fed mice chow only. Feeding a glycolipid synthesis inhibitor (D-PDMP) daily by oral gavage prevented cardiac hypertrophy, prevented the aortic intima media thickening, and reversed the blood pressure to normal level. Thus, glycolipid synthesis plays a central role in cardiac pathophysiology in transgenic apoE−/− mice. These findings point to novel drug targets to ameliorate heart diseases by manipulating glycolipid synthesis.

Accordingly, in one aspect, the present invention provides methods and compositions useful for treating or preventing cardiac hypertrophy. In a specific embodiment, a method for treating or preventing cardiac hypertrophy in a patient comprises the step of administering a therapeutically effective amount of a glycolipid synthesis inhibitor. In certain embodiments, the glycolipid synthesis inhibitor is a small molecule, an antibody, a protein, a peptide, or a nucleic acid. In one embodiment, the glycolipid synthesis inhibitor inhibits glucosylceramide synthase. In another embodiment, the glycolipid synthesis inhibitor inhibits lactosylceramide synthase. In a specific embodiment, the glycolipid synthesis inhibitor is D-threo-1-Phenyl-2-decanoylamino-3-morpholino-1-propanol HCl (D-PDMP). In other embodiments, the glycolipid synthesis inhibitor is a D-PDMP analog. The D-PDMP analog can be selected from the group consisting of D-threo-1-(3′,4′-ethyleneidoxy)phenyl-2-palmitoylaminod-3-pyrrolidino-1-propanol (EtDO-P4), D-threo-4′-hydroxy-1-phenyl-2-palmitoylamino-3-pyrrolidino-1-propanol (pOH-P4), D-threo-1-(3′,4′-trimethylenedioxy)phenyl-2-palmitoylamino-3-pyrrolidino-1-propanol (trimethylenedioxy-P4), and D-threo-1-(3′,4′-methylenedioxy)phenyl-2-palmitoylamino-3-pyrrolidino-1-propanol (methylenedioxy-P4).

In other embodiments, the glycolipid synthesis inhibitor is 1,5-(butylimino)-1,5-dideoxy-D-glucitol (NB-DNJ). In further embodiments, the glycolipid synthesis inhibitor is a NB-DNJ analog. The D-PDMP analog can be selected from the group consisting of N-nonyldeoxynojirimycin (NN-DNJ), N-butyl-deoxygalatconojirimycin (NB-DGJ), adamantan-1-yl glucosyl ceramide, (1R,2R) nonanoic acid[2-3-(2′,3′-dihydro-benzo[1,4]dioxin-6-′yl)-2-hydroxy-1-pyrrolidin-1-ylmethyl-ethyl]-1 amide-L-tartaric acid salt (Genz-1223346) and N-(5-adamantane-1-yl-methoxy)pentyl)-deoxynojirimycin (AMP-DNJ or AMP-DNM).

The present invention also provides a method for treating or preventing cardiac hypertrophy in a patient comprising the step of administering an effective amount of a glucosylceramide synthase inhibitor. Alternatively, or in combination, a method for treating or preventing cardiac hypertrophy in a patient comprises the step of administering an effective amount of a lactosylceramide synthase inhibitor. In another embodiment, a method for treating or preventing cardiac hypertrophy and/or aortic intimal media thickening in a patient comprises the step of administering a therapeutically effective amount of a glycolipid synthesis inhibitor. In a further embodiment, a method for treating or preventing aortic intimal media thickening in a patient comprises the step of administering a therapeutically effective amount of a glycolipid synthesis inhibitor. In yet another embodiment, a method for treating or preventing atherosclerosis in a patient comprises the step of administering a therapeutically effective amount of a glycolipid synthesis inhibitor.

In another aspect, the present invention provides diagnostic methods utilizing a glycosyltransferase biomarker. In one embodiment, a method for diagnosing cardiac hypertrophy in a patient comprises the steps of (a) obtaining biological sample from the patient; (b) determining the level of expression of a glycosyltransferase biomarker in the sample; and (c) diagnosing the patient has having cardiac hypertrophy if the level of expression of the glycosyltransferase biomarker is significantly increased relative to a baseline level of the biomarker that is indicative of not having cardiac hypertrophy. In a specific embodiment, the determining step is performed using mass spectrometry. In another specific embodiment, the determining step is performed using an immunoassay.

In other embodiments, a method for diagnosing cardiac hypertrophy in a patient comprises the steps of (a) determining the level of expression of a glycosyltransferase biomarker in a sample obtained from the patient; (b) comparing the level of expression of the biomarker in the sample to reference levels of the biomarker that correlate to having cardiac hypertrophy and not having cardiac hypertrophy using a classification algorithm; and (c) diagnosing the patient has having cardiac hypertrophy if the level of expression of the glycosyltransferase biomarker correlates to a reference level of the biomarker that is indicative of not having cardiac hypertrophy. The determining step can be performed using an immunoassay or mass spectrometry.

In particular embodiments, a method for treating cardiac hypertrophy in a patient comprises the steps of (a) determining the level of expression of a glycosyltransferase biomarker in a sample obtained from the patient; (b) comparing the level of expression of the biomarker in the sample to reference levels of the biomarker that correlate to having cardiac hypertrophy and not having cardiac hypertrophy using a classification algorithm; and (c) treating the patient with an appropriate cardiac hypertrophy treatment if the level of expression of the glycosyltransferase biomarker correlates to a reference level of the biomarker that is indicative of not having cardiac hypertrophy.

In alternative embodiments, a method for diagnosing cardiac hypertrophy in a patient comprises the steps of (a) measuring the levels of one or more modified and/or unmodified glycosyltransferase proteins in a sample collected from the patient; and (b) comparing the levels of the measured one or more modified and/or unmodified glycosyltransferase proteins with predefined levels of the same proteins that correlate to a patient having cardiac hypertrophy and predefined levels of the same proteins that correlate to a patient not having cardiac hypertrophy, wherein a correlation to one of the predefined levels provides the diagnosis. The modifications can include, but are not limited to, glycosylation, phosphorylation, methylation and acetylation.

In other embodiments, a method for diagnosing cardiac hypertrophy in a patient comprises the steps of (a) measuring the levels of one or more post-translationally modified and unmodified glycosyltransferase peptides in a sample collected from the patient using mass spectrometry; (b) comparing the levels of the measured one or more post-translationally modified glycosyltransferase peptides to the levels of the measured one or more unmodified glycosyltransferase peptides; and (c) correlating the compared levels to a patient having cardiac hypertrophy or to a patient not having cardiac hypertrophy, thereby providing the diagnosis. In specific embodiments, the mass spectrometry measurement step is performed using multi-chain reaction (MRM) mass spectrometry (MRM-MS) with liquid chromatography (LC)-trandem MS.

In certain embodiments, a method for diagnosing cardiac hypertrophy in a patient comprises the steps of (a) measuring the levels of one or more post-translationally modified and unmodified glycosyltransferase peptides in a sample collected from the patient using MRM-MS; and (b) comparing the ratio of the measured one or more post-translationally modified glycosyltransferase peptides and the measured one or more unmodified glycosyltransferase peptides to predefined ratios of the same modified/unmodified glycosyltransferase that correlate to a patient having cardiac hypertrophy and predefined ratios of the same modified/unmodified glycosyltransferase that correlate to a patient not having cardiac hypertrophy, wherein a correlation to one of the predefined levels provides the diagnosis.

### BRIEF DESCRIPTION OF THE FIGURES

FIG. 1 presents the experimental design using apolipoprotein E−/− mice to determine the role of glycolipids and glycosyltransferase in atherosclerosis and cardiac hypertrophy.

FIG. 2 shows the effects of feeding a hyperlipidemic diet with/without 5 and 10 mpk D-PDMP on the activity of lactosylceramide synthase (FIG. 2A) and glucosylceramide synthase (FIG. 2B) in aorta tissue in 20 week old apoE−/− mice. The activity of lactosylceramide synthase and glucosylceramide synthase increased in placebo mouse aorta compared to control. Treatment with D-PDMP dose-dependently decreased the activity of lactosylceramide synthase. But a D-PDMP dose-dependent decrease in glucosylceramide synthase activity was not observed.

FIG. 3A displays a 2D-mode ultrasound (US) images from a normal control (A) and an Apo E−/− mouse on high fatty diet 2% fat and 1.25 cholesterol, serving as placebo (B), high fatty diet plus 10 mpk of D-PDMP (C) and 5 mpk (D) at 20 weeks of age. The arrows in the control mouse images indicate normal wall thickness as compared to increased wall thickness in placebo treated mice. Aortic Intima Media Thickening (AoIMT). **p<0.001 ***p<0.0001, n=5. FIG. 3B is a graphical representation of aortic intima wall thickness (AoIMT) of baseline at 12 weeks of age, Control, D-PDMP 10 MPK+Fat, D-PDMP 5 MPK+Fat, Placebo (Vehicle+Fat fed) Apo E−/− mice at 16 and 20 weeks of age. The AoIMT in the placebo mice increased significantly as compared to control and 5 MPK treated mice. Control and 10 MPK treated mice were not significantly different in 16 and 20 week old mice and when compared to baseline (mouse age, 12 weeks). AoIMT progressively increased in 5 MPK and Placebo groups in 16 and 20 week old mice. However, AoMT in the 10 MPK fed mice did not change.

FIG. 4A shows M mode (A, B, C, D) echocardiography analysis of Control, Placebo and D-PDMP fed Apo E−/− mice treated for up to 20 weeks of age. A significant decline of left ventricle end systolic dimension (LVESD) (see trace) suggests reduction in myocardial contractility (B, D). Inter-ventricular septal thickness at end diastolic phase (IVESD) and posterior wall thickness at end diastolic phase (PWTED) are thicker in placebo and 5-MPK D-PDMP+Fat fed mice suggesting hypertrophied wall as compared to non hypertrophied Control and 10 MPK+Fat treated mice. Left ventricle end systolic dimension (LVESD), left ventricle end diastolic dimension (LVEDD), inter-ventricular septal thickening at end diastolic (IVSD) and posterior wall thickness at end diastolic phase (PWTED). p<0.001**, p<0.0001***, n=5. FIG. 4B is a graphical representation of Fractional shortening (FS %) indexing cardiac contractility representative of baseline at 12 weeks of age, Control, Placebo, D-PDMP 10 MPK+Fat, and D-PDMP 5 MPK+Fat diet in Apo E−/− mice treated from 12 weeks to 16 and 20 weeks of age. A significant decline in FS % of left ventricle suggest reduction in myocardial contractility in placebo and 5 MPK treated mice at 16 and 20 weeks of age. Ten MPK D-PDMP fed mice did not significantly differ from the baseline and control at 16 and 20 weeks, indicating that the D-PDMP is cardio-protective. p<0.001**, p<0.0001***, n=5.

FIG. 5 shows that the glycolipid synthesis inhibitor D-PDMP reduced intimal wall thickness, fibrosis and plaque accumulation.

FIG. 6 is a table showing that D-PDMP reduced cardiovascular thickening, plaque accumulation, blood pressure and prevented arterial stiffness.

FIG. 7. Treatment with glycosphingolipid synthesis inhibitor prevents cardiac hypertrophy in apoE−/− mice. (a) M-mode echocardiogram or ultrasound (US) images from an Apo E−/− mice fed chow alone (A), high fat diet of 2% fat and 1.25 cholesterol (HFHC) plus 10 mpk of D-PDMP (B) and HFHC plus vehicle (C) fed mice. The bars indicate left ventricle dimension and wall thickness at end diastolic and end systolic phase. The placebo or the vehicle+fat treated mice show a significant increase in wall thickness and significant decline in cardiac contractility. This is consistent with an increase in left ventricle mass (LV mass) and the decline in fractional shortening (FS) data, respectively. (b) The gross anatomy of the heart bellow further confirms the heart size. (c) Fraction shortening. (d) Left ventricular mass.

FIG. 8. Treatment with glycosphingolipid synthesis inhibitor ameliorates cardiac hypertrophy induced by trans-aortic constriction. M-mode echocardiogram showing control or Sham, trans-aortic constricted (TAC) and TAC plus 10 mpk of D-PDMP fed mice 1-6 weeks after surgery. (b) Gross anatomy of the control or sham, TAC and TAC+10 mpk of D-PDMP treated mice hearts. (c) Graphical representation of left ventricle fractional shortening (FS) and (d) mass (LV mass) at baseline, the 1^{st}, 2^{nd} and 6^{th} week after TAC.

FIG. 9. D-PDMP inhibits the activity of glycosphingolipid glycosysltransferase activity in the heart tissue in apoE−/− mice. Feeding 5 mpk and 10 mpk of D-PDMP for 20 weeks (FIGS. 3A, B) and 36 weeks (FIG. 3C, D) dose-dependently decreased the activity of glucosylceramide synthase and lactosylceramide synthase.

FIG. 10. D-PDMP inhibits the mass of glycosphingolipids in the heart tissue in apoE−/− mice.

FIG. 11. D-PDMP decreases the level of glycosphingolipids in mice subject to trans-aortic constriction.

FIG. 12. Treatment with D-PDMP ameliorates the expression of genes implicated in cardiac hypertrophy. Treatment with 10 mpk Drug (PDMP) blunts cardiac hypertrophic marker genes expression in left ventricular tissues from (a) Apo E−/− placebo and (b) TAC mice. RNA was extracted from ventricular tissues of mice from different experimental groups. Open Array analysis was performed using Quant Studio 12K Flex Real Time PCR. For RT-PCR reactions (20 μl), equal amount (500 ng) of total RNA was used. Expression levels of 18S RNA, beta actin and GAPDH were used to normalize for variations in the amount of RNA. RNA expression levels were quantified by using Expression suite software. 10 mpk drug blunted the ANP, BNP and βMHC mRNA expression levels induced in TAC and Apo E−/− mice (fed with high fat and cholesterol diet).

FIG. 13. Treatment with 10 mpk Drug (PDMP) induces antioxidant defence in hypertrophic heart in vivo. Treatment with 10 mpk Drug (PDMP) induces antioxidant defence in left ventricular tissues of Apo E−/− placebo and TAC mice. RNA was extracted from ventricular tissues of mice from different experimental groups. Open Array analysis was performed using Quant Studio 12K Flex Real Time PCR. For RT-PCR reactions (20 μl), equal amount (500 ng) of total RNA was used. Expression levels of 18S RNA, beta actin and GAPDH were used to normalize for variations in the amount of RNA. RNA expression levels were quantified by using Expression suite software. 10 mpk drug induces the SOD1, SOD2, Catalase mRNA expression levels both in TAC and Apo E−/− mice (fed with high fat and cholesterol diet). HIF-1α expression was increased in hearts from Apo E−/− placebo and TAC animals compared with 10 mpk Drug treated group. (a) Apo E−/− placebo and (b) TAC mice.

FIG. 14. Treatment with 10 mpk Drug (PDMP) decreased the expression of genes involved in collagen synthesis in hypertrophic heart in vivo. Collagen and MMP gene levels in left ventricular tissues from TAC studies. RNA was extracted from ventricular tissues of mice from different experimental groups. Open Array analysis was performed using Quant Studio 12K Flex Real Time PCR. For RT-PCR reactions (20 μl), equal amount (500 ng) of total RNA was used. Expression levels of 18S RNA, beta actin and GAPDH were used to normalize for variations in the amount of RNA. RNA expression levels were quantified by using Expression suite software. 10 mpk drug decreased the mRNA levels of collagen type II and III genes overexpressed in TAC mice.

FIG. 15. Real Time quantitative PCR analysis of alpha skeletal actin and beta MHC expression in Ventricular Mice heart tissues.

FIG. 16. TGFβ over expression in hypertrophic mouse heart is mitigated by the use of D-PDMP. Left ventricular heart homogenates were prepared from placebo, 5 mpk and 10 mpk treated mouse hearts. Western blot analysis was done using anti TGFβ antibody. Expression levels of GAPDH were used to normalize the protein expression. Expression levels were quantified by using Image J v.1.45s (NIH, USA) software. (a) Western immunoblot of TGF-β. (b) Corresponding densitomeric scan.

FIG. 17. D-PDMP decreases the expression of p44MAPK. The heart tissue lysates were separated by SDS gel electrophoresis, blotted onto nitro cellulose, and p44MAPK detected using a polyclonal antibody against this antigen. GAPDH served as a house keeping protein. Note a marked increase in the phosphorylated form of p44MAPK (indicated by an arrow) in placebo heart tissue as compared to control. And a decrease in the level of this protein kinase in mice treated with 10 mpk of D-PDMP. (a) Western immunoblot of pP44MAPK. (b) Corresponding densitomeric scan.

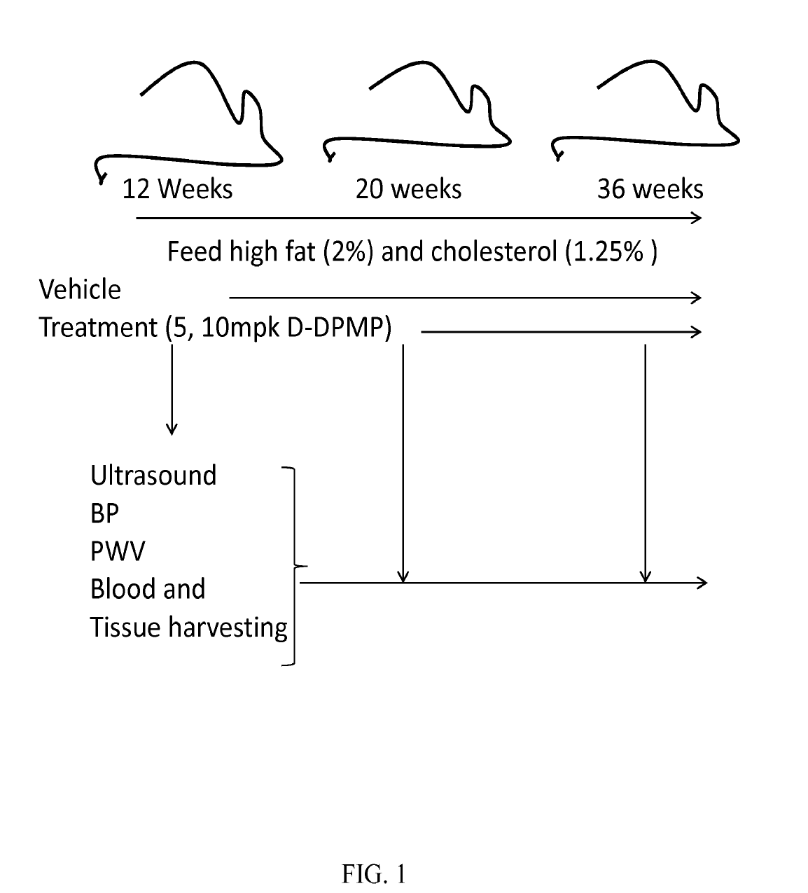

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. DEFINITIONS

The following definitions are used throughout this specification. Other definitions are embedded within the specification for ease of reference.

The term “glycolipid synthesis inhibitor” refers to an agent that inhibits the synthesis or biological activity of a glycolipid. Glycolipid synthesis inhibitors are known in the art and include, but are not limited to, D-PDMP, NB-DNJ, and the like. The term also includes compounds that have activity in addition to glycolipid synthesis inhibitory activity. The term is used interchangeably with “glycosphingolipid synthesis inhibitor,” and the like.

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of cardiac hypertrophy. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cerebrospinal fluid, urine, saliva, stool and synovial fluid), solid tissue samples such as a biopsy specimen or tissue cultures or cells derived therefrom and the progeny thereof. In a specific embodiment, a sample comprises a blood sample. In another embodiment, a sample comprises a plasma sample. In yet another embodiment, a serum sample is used.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

As used herein, the term “cardiac hypertrophy” is used in its ordinary meaning as understood by the medical community. It generally refers to the process in which adult cardiac myocytes respond to stress through hypertrophic growth. Such growth is characterized by cell size increases without cell division or proliferation, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the term “cardiovascular disease” refers to diseases and disorders of the heart and circulatory system. Exemplary cardiovascular diseases, including cholesterol- or lipid-related disorders, include, but are not limited to acute coronary syndrome, angina, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, and transitory ischemic attacks.

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The terms “heart failure,” “manifestations of heart failure,” “symptoms of heart failure,” and the like are used broadly to encompass all of the sequelae associated with heart failure, such as shortness of breath, pitting edema, an enlarged tender liver, engorged neck veins, pulmonary rales and the like including laboratory findings associated with heart failure.

As used herein, “atherosclerosis” denotes a disease affecting arterial blood vessels. Atherosclerosis can be characterized by a chronic inflammatory response in the walls of arteries, mainly due to the accumulation of macrophages and promoted by low density lipoproteins without adequate removal of fats and cholesterol from macrophages by functional high density lipoproteins.

As used herein, the term “comparing” refers to making an assessment of how the proportion, level or cellular localization of one or more biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard or control sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels/ratios that correspond to, for example, a patient having cardiac hypertrophy, not having cardiac hypertrophy, is responding to treatment for cardiac hypertrophy, is not responding to treatment for cardiac hypertrophy, is/is not likely to respond to a particular cardiac hypertrophy treatment, or having/not having another disease or condition. In a specific embodiment, the term “comparing” refers to assessing whether the level of one or more biomarkers of the present invention in a sample from a patient is the same as, more or less than, different from other otherwise correspond (or not) to levels/ratios of the same biomarkers in a control sample (e.g., predefined levels/ratios that correlate to uninfected individuals, standard cardiac hypertrophy levels/ratios, etc.).

In another embodiment, the term “comparing” refers to making an assessment of how the proportion, level or cellular localization of one or more biomarkers in a sample from a patient relates to the proportion, level or cellular localization of another biomarker in the same sample. For example, a ratio of one biomarker to another from the same patient sample can be compared. In another embodiment, a level of one biomarker in a sample (e.g., an unmodified biomarker protein) can be compared to the level of the same biomarker (e.g., a modified biomarker protein) in the sample. In a specific embodiment, the proportion of a modified biomarker protein can be compared to the unmodified protein, both of which are measured in the same patient sample. Ratios of modified:unmodified biomarker proteins can be compared to other protein ratios in the same sample or to predefined reference or control ratios. In a specific embodiment, different isoforms of glycosyltransferase can be measured. In addition or alternatively, a modified glycosyltransferase may comprise a post-translationally modified (PTM) form. In such embodiments, a glycosyltransferase protein/peptide may have undergone PTM due to cardiac hypertrophy, fibrosis or other condition.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has cardiac hypertrophy. In specific embodiments, the parameter may comprise the level of one or more biomarkers of the present invention. A particular set or pattern of the amounts of one or more biomarkers may indicate that a patient has a cardiac hypertrophy (i.e., correlates to a patient having cardiac hypertrophy). In other embodiments, a correlation could be the ratio of a modified protein to the unmodified protein indicates (or a change in the ratio over time or as compared to a reference/control ratio) could mean that the patient has cardiac hypertrophy).

In other embodiments, a particular set or pattern of the amounts of one or more biomarkers may be correlated to a patient being unaffected (i.e., indicates a patient does not have cardiac hypertrophy). In certain embodiments, “indicating,” or “correlating,” as used according to the present invention, may be by any linear or non-linear method of quantifying the relationship between levels/ratios of biomarkers to a standard, control or comparative value for the assessment of the diagnosis, prediction of cardiac hypertrophy or cardiac hypertrophy progression, assessment of efficacy of clinical treatment, identification of a patient that may respond to a particular treatment regime or pharmaceutical agent, monitoring of the progress of treatment, and in the context of a screening assay, for the identification of a cardiac hypertrophy therapeutic.

The terms “measuring” and “determining” are used interchangeably throughout, and refer to methods which include obtaining a patient sample and/or detecting the level of a biomarker(s) in a sample. In one embodiment, the terms refer to obtaining a patient sample and detecting the level of one or more biomarkers in the sample. In another embodiment, the terms “measuring” and “determining” mean detecting the level of one or more biomarkers in a patient sample. Measuring can be accomplished by methods known in the art and those further described herein. The term “measuring” is also used interchangeably throughout with the term “detecting.”

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed for levels/ratios in a sample from an unaffected individual (UI) or a normal control individual (NC) (both terms are used interchangeably herein). In another embodiment, a “suitable control” or “appropriate control” is a value, level, feature, characteristic, property, ratio, etc. determined prior to performing a therapy (e.g., a cardiac hypertrophy treatment) on a patient. In yet another embodiment, a transcription rate, mRNA level, translation rate, protein level/ratio, biological activity, cellular characteristic or property, genotype, phenotype, etc., can be determined prior to, during, or after administering a therapy into a cell, organ, or patient. In a further embodiment, a “suitable control” or “appropriate control” is a predefined value, level, feature, characteristic, property, ratio, etc. A “suitable control” can be a profile or pattern of levels/ratios of one or more biomarkers of the present invention that correlates to cardiac hypertrophy, to which a patient sample can be compared. The patient sample can also be compared to a negative control, i.e., a profile that correlates to not having cardiac hypertrophy.

As used herein, the term “effective,” means adequate to accomplish a desired, expected, or intended result. More particularly, a “therapeutically effective amount” as provided herein refers to an amount of a glycolipid synthesis inhibitor of the present invention, either alone or in combination with another therapeutic agent, necessary to provide the desired therapeutic effect, e.g., an amount that is effective to prevent, alleviate, or ameliorate symptoms of disease or prolong the survival of the subject being treated. In a specific embodiment, the term “therapeutically effective amount” as provided herein refers to an amount of a glycolipid synthesis inhibitor, necessary to provide the desired therapeutic effect, e.g., an amount that is effective to prevent, alleviate, or ameliorate symptoms of disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies. In specific embodiments, antibodies may be raised against glycosyltransferases (e.g., glucosylceramide synthase and lactosylceramide synthase) and used as glycolipid synthesis inhibitors. In other embodiments, antibodies may be raised against glycoyltransferase protein/peptides that have undergone post-translational modification(s) due to cardiac hypertrophy.

The terms “specifically binds to,” “specific for,” and related grammatical variants refer to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

Optional” or “optionally” means that the subsequently described event or circumstance can or cannot occur, and that the description includes instances where the event or circumstance occurs and instances where it does not.

As used herein, a “subject” or “patient” means an individual and can include domesticated animals, (e.g., cats, dogs, etc.); livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. In particular, the term also includes mammals diagnosed with cardiac hypertrophy.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

### II. GLYCOPLIPID SYNTHESIS INHIBITORS

The present invention utilized glycolipid synthesis inhibitors. In certain embodiments, the glycolipid synthesis inhibitor is D-threo-1-Phenyl-2-decanoylamino-3-morpholino-1-propanol HCl (D-PDMP). D-PDMP is a glucosylceramide synthase and lactosylceramide synthase inhibitor. The molecular formula for D-PDMP is C₂₃H₃₈N₂₀₃HCl. D-PDMP includes a molecular weight of 427.1 and is soluble in water. A group of inhibitors have been developed using PDMP as the lead compound. The parent compound of the new group of inhibitors, D-threo-1-phenyl-2-palmitoylamino-3-pyrrolidino-1-propanol (P4) can be used in the present invention. More specifically, the P4 analogs, D-threo-1-(3′,4′-ethyleneidoxy)phenyl-2-palmitoylaminod-3-pyrrolidino-1-propanol (EtDO-P4) and D-threo-4′-hydroxy-1-phenyl-2-palmitoylamino-3-pyrrolidino-1-propanol (pOH-P4) can be used (effective concentration (ID₅₀) of about 0.1 μM). Other P4 derivatives include D-threo-1-(3′,4′-trimethylenedioxy)phenyl-2-palmitoylamino-3-pyrrolidino-1-propanol (trimethylenedioxy-P4), D-threo-1-(3′,4′-methylenedioxy)phenyl-2-palmitoylamino-3-pyrrolidino-1-propanol (methylenedioxy-P4). Dosages of P4 derivatives including EtDO-P4 are easily determined by the skilled artisan. Such dosages may range from about 0.5 mg/kg to about 50 mg/kg, from about 1 mg/kg to about 10 mg/kg by intraperitoneal or equivalent administration from one to five times daily. Such dosages may range from about 5 mg/kg to about 5 g/kg, from about 10 mg/kg to about 1 g/kg by oral or equivalent administration from one to five times daily. A particularly oral dose range for a P4-like compound is from about 6 mg/kg/day to about 600 mg/kg/day.

In other embodiments, the glycolipid synthesis inhibitor is 1,5-(butylimino)-1,5-dideoxy-D-glucitol (also known as N-butyldeoxynojirimycin (NB-DNJ), Miglustat or Zavesca®) (effective concentration (ID₅₀ of about 10-100 μM) (Actelion Pharmaceuticals U.S., Inc. (San Francisco, Calif.)). NB-DNJ is an inhibitor of glucosylceramide synthase, a glucosyl transferase enzyme that plays a role in the synthesis of many glycosphingolipids. Miglustat is soluble in water. The molecular formula for Miglustat is C₁₀H₂₁NO₄ and has a molecular weight of 219.28. In other specific embodiments, N-nonyldeoxynojirimycin (NN-DNJ) can be used. In yet another embodiment, the galactose analogue N-butyl-deoxygalatconojirimycin (NB-DGJ) can be used. Another particular deoxynojirimycin derivative for use in the combination therapies of the invention is N-(5-adamantane-1-yl-methoxy)pentyl)-deoxynojirimycin (AMP-DNJ or AMP-DNM). Other inhibitors include adamantan-1-yl glucosyl ceramide, (1R,2R) nonanoic acid[2-3-(2′,3′-dihydro-benzo[1,4]dioxin-6-′yl)-2-hydroxy-1-pyrrolidin-1-ylmethyl-ethyl]-1 amide-L-tartaric acid salt (Genz-1223346). Dosages of DNJ derivatives including NB-DNJ, NB-DGJ, AMP-DNJ in are also readily determined by the skilled artisan. Such dosages may range from about 0.01 mg/kg to about 1000 mg/kg, from about 0.1 mg/kg to about 100 mg/kg, more specifically from about 1 mg/kg to about 10 mg/kg, by intraperitoneal or equivalent administration from one to five times daily. Such dosages, when administered orally, may range from two- to twenty-fold greater. For example, NB-DNJ has been administered orally to humans in a 100 mg dose three times per day for twelve months, and a daily dose of up to 3 gm has been used. A particular oral dose range for a DNJ-like compound is from about 60 mg/kg/day to about 900 mg/kg/day.

### III. OTHER GLYCOLIPID SYNTHESIS INHIBITORS

A. RNA Interference Compositions for Targeting Glyscosyltransferase mRNAs

In one aspect of the present invention, the expression of glycosyltransferases may be inhibited by the use of RNA interference techniques (RNAi). RNAi is a remarkably efficient process whereby double-stranded RNA (dsRNA) induces the sequence-specific degradation of homologous mRNA in animals and plant cells. See Hutvagner and Zamore, 12 CURR. OPIN. GENET. DEV. 225-32 (2002); Hammond et al., 2 NATURE REV. GEN. 110-19 (2001); Sharp, 15 GENES DEV. 485-90 (2001). RNAi can be triggered, for example, by nucleotide (nt) duplexes of small interfering RNA (siRNA) (Chiu et al., 10 MOL. CELL. 549-61 (2002); Elbashir et al., 411 Nature 494-98 (2001)), micro-RNAs (miRNA), functional small-hairpin RNA (shRNA), or other dsRNAs which are expressed in-vivo using DNA templates with RNA polymerase III promoters. See, e.g., Zeng et al., 9 MOL. CELL. 1327-33 (2002); Paddison et al., 16 GENES DEV. 948-58 (2002); Lee et al., 20 NATURE BIOTECHNOL. 500-05 (2002); Paul et al., 20 NATURE BIOTECHNOL. 505-08 (2002); Tuschl, 20 NATURE BIOTECHNOL. 440-48 (2002); Yu et al., 99(9) PROC. NATL. ACAD. SCI. USA, 6047-52 (2002); McManus et al., 8 RNA 842-50 (2002); Sui et al., 99(6) PROC. NATL. ACAD. SCI. USA 5515-20 (2002).

1. Small Interfering RNA

In particular embodiments, the present invention features “small interfering RNA molecules” (“siRNA molecules” or “siRNA”), methods of making siRNA molecules and methods for using siRNA molecules (e.g., research and/or therapeutic methods). The siRNAs of this invention encompass any siRNAs that can modulate the selective degradation of glycosyltransferase mRNA.

In a specific embodiment, the siRNA of the present invention may comprise double-stranded small interfering RNA molecules (ds-siRNA). A ds-siRNA molecule of the present invention may be a duplex made up of a sense strand and a complementary antisense strand, the antisense strand being sufficiently complementary to a target glycosyltransferase mRNA to mediate RNAi. The siRNA molecule may comprise about 10 to about 50 or more nucleotides. More specifically, the siRNA molecule may comprise about 16 to about 30, e.g., 16, 17, 18, 19, 20, 21, 22, 23, 24, 25, 26, 27, 28, 29, or 30 nucleotides in each strand. The strands may be aligned such that there are at least 1, 2, or 3 bases at the end of the strands which do not align (e.g., for which no complementary bases occur in the opposing strand) such that an overhang of 1, 2 or 3 residues occurs at one or both ends of the duplex when strands are annealed.

In an alternative embodiment, the siRNA of the present invention may comprise single-stranded small interfering RNA molecules (ss-siRNA). Similar to the ds-siRNA molecules, the ss-siRNA molecule may comprise about 10 to about 50 or more nucleotides. More specifically, the ss-siRNA molecule may comprise about 15 to about 45 or more nucleotides. Alternatively, the ss-siRNA molecule may comprise about 19 to about 40 nucleotides. The ss-siRNA molecules of the present invention comprise a sequence that is “sufficiently complementary” to a target mRNA sequence to direct target-specific RNA interference (RNAi), as defined herein, e.g., the ss-siRNA has a sequence sufficient to trigger the destruction of the target mRNA by the RNAi machinery or process. In one embodiment, the ss-siRNA molecule can be designed such that every residue is complementary to a residue in the target molecule. Alternatively, substitutions can be made within the molecule to increase stability and/or enhance processing activity of the molecule. Substitutions can be made within the strand or can be made to residues at the ends of the strand. In a specific embodiment, the 5′-terminus may be phosphorylated (e.g., comprises a phosphate, diphosphate, or triphosphate group). In another embodiment, the 3′ end of an siRNA may be a hydroxyl group in order to facilitate RNAi, as there is no requirement for a 3′ hydroxyl group when the active agent is a ss-siRNA molecule. In other instances, the 3′ end (e.g., C3 of the 3′ sugar) of ss-siRNA molecule may lack a hydroxyl group (e.g., ss-siRNA molecules lacking a 3′ hydroxyl or C3 hydroxyl on the 3′ sugar (e.g., ribose or deoxyribose).

In another aspect, the siRNA molecules of the present invention may be modified to improve stability under in vitro and/or in vivo conditions, including, for example, in serum and in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

Furthermore, the siRNAs of the present invention may include modifications to the sugar-phosphate backbone or nucleosides. These modifications can be tailored to promote selective genetic inhibition, while avoiding a general panic response reported to be generated by siRNA in some cells. In addition, modifications can be introduced in the bases to protect siRNAs from the action of one or more endogenous enzymes.

In an embodiment of the present invention, the siRNA molecule may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues. Examples of nucleotide analogues include sugar- and/or backbone-modified ribonucleotides (e.g., include modifications to the phosphate-sugar backbone). For example, the phosphodiester linkages of natural RNA may be modified to include at least one of a nitrogen or sulfur heteroatom. In backbone-modified ribonucleotides, the phosphoester group connecting to adjacent ribonucleotides may be replaced by a modified group, e.g., a phosphothioate group. In sugar-modified ribonucleotides, the 2′ OH-group may be replaced by a group selected from H, OR, R, halo, SH, SR, NH₂, NHR, NR₂ or ON, wherein R is C₁-C₆ alkyl, alkenyl or alkynyl and halo is F, Cl, Br or I.

Nucleobase-modified ribonucleotides may also be utilized, e.g., ribonucleotides containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

Derivatives of siRNAs may also be utilized herein. For example, cross-linking can be employed to alter the pharmacokinetics of the composition, e.g., to increase half-life in the body. Thus, the present invention includes siRNA derivatives that include siRNA having two complementary strands of nucleic acid, such that the two strands are crosslinked. The present invention also includes siRNA derivatives having a non-nucleic acid moiety conjugated to its 3′ terminus (e.g., a peptide), organic compositions (e.g., a dye), or the like. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The siRNAs of the present invention can be enzymatically produced or totally or partially synthesized. Moreover, the siRNAs can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although one hundred percent (100%) sequence identity between the siRNA and the target region is preferred in particular embodiments, it is not required to practice the invention. siRNA molecules that contain some degree of modification in the sequence can also be adequately used for the purpose of this invention. Such modifications may include, but are not limited to, mutations, deletions or insertions, whether spontaneously occurring or intentionally introduced.

Moreover, not all positions of a siRNA contribute equally to target recognition. In certain embodiments, for example, mismatches in the center of the siRNA may be critical and could essentially abolish target RNA cleavage. In other embodiments, the 3′ nucleotides of the siRNA do not contribute significantly to specificity of the target recognition. In particular, residues 3′ of the siRNA sequence which is complementary to the target RNA (e.g., the guide sequence) may not critical for target RNA cleavage.

Sequence identity may be determined by sequence comparison and alignment algorithms known to those of ordinary skill in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (e.g., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (e.g., a local alignment). A non-limiting example of a local alignment algorithm utilized for the comparison of sequences is the algorithm of Karlin and Altschul, 87 PROC. NATL. ACAD. SCI. USA 2264-68 (1990), and as modified as in Karlin and Altschul 90 PROC. NATL. ACAD. SCI. USA 5873-77 (1993). Such an algorithm is incorporated into the BLAST programs (version 2.0) of Altschul, et al., 215 J. MOL. BIOL. 403-10 (1990).

In another embodiment, the alignment may optimized by introducing appropriate gaps and determining percent identity over the length of the aligned sequences (e.g., a gapped alignment). To obtain gapped alignments for comparison purposes, Gapped BLAST can be utilized as described in Altschul et al., 25(17) NUCLEIC ACIDS RES. 3389-3402 (1997). In another embodiment, the alignment may be optimized by introducing appropriate gaps and determining percent identity over the entire length of the sequences aligned (e.g., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In particular embodiments, greater than 90% sequence identity, e.g., 91%, 92%, 93%, 94%, 95%, 96%, 97%, 98%, 99% or even 100% sequence identity, between the siRNA and the portion of the target gene may be used. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional hybridization conditions include, but are not limited to, hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length can be about 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na⁺])+0.41(% G+C)-(600/N), where N is the number of bases in the hybrid, and [Na⁺] is the concentration of sodium ions in the hybridization buffer ([Na⁺] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 50 or more bases.

2. Other Compositions for Targeting Glycosyltransferase DNA or mRNA

Antisense molecules can act in various stages of transcription, splicing and translation to block the expression of a target gene. Without being limited by theory, antisense molecules can inhibit the expression of a target gene by inhibiting transcription initiation by forming a triple strand, inhibiting transcription initiation by forming a hybrid at an RNA polymerase binding site, impeding transcription by hybridizing with an RNA molecule being synthesized, repressing splicing by hybridizing at the junction of an exon and an intron or at the spliceosome formation site, blocking the translocation of an mRNA from nucleus to cytoplasm by hybridization, repressing translation by hybridizing at the translation initiation factor binding site or ribosome biding site, inhibiting peptide chain elongation by hybridizing with the coding region or polysome binding site of an mRNA, or repressing gene expression by hybridizing at the sites of interaction between nucleic acids and proteins. An example of an antisense oligonucleotide of the present invention is a cDNA that, when introduced into a cell, transcribes into an RNA molecule having a sequence complementary to at least part of the glycosyltransferase mRNA.

Furthermore, antisense oligonucleotides of the present invention include oligonucleotides having modified sugar-phosphodiester backbones or other sugar linkages, which can provide stability against endonuclease attacks. The present invention also encompasses antisense oligonucleotides that are covalently attached to an organic or other moiety that increase their affinity for a target nucleic acid sequence. For example, intercalating agents, alkylating agents, and metal complexes can be also attached to the antisense oligonucleotides of the present invention to modify their binding specificities.

The present invention also provides ribozymes as a tool to inhibit glycosyltransferase expression. Ribozymes are enzymatic RNA molecules capable of catalyzing the specific cleavage of RNA. The characteristics of ribozymes are well-known in the art. See, e.g., Rossi, 4 CURRENT BIOLOGY 469-71 (1994). Without being limited by theory, the mechanism of ribozyme action involves sequence specific hybridization of the ribozyme molecule to complementary target RNA, followed by an endonucleolytic cleavage. In particular embodiments, the ribozyme molecules include one or more sequences complementary to the target gene mRNA, and include the well known catalytic sequence responsible for mRNA cleavage. See U.S. Pat. No. 5,093,246. Using the known sequence of the target glycosyltransferase mRNA, a restriction enzyme-like ribozyme can be prepared using standard techniques.

The expression of a glycosyltransferase gene can also be inhibited by using triple helix formation. Nucleic acid molecules to be used in triple helix formation for the inhibition of transcription can be single stranded and composed of deoxynucleotides. The base composition of these oligonucleotides must be designed to promote triple helix formation via Hoogsteen base paring rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules that are purine-rich, e.g., containing a stretch of G residues, may be chosen. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair first with one strand of a duplex and then the other, eliminating the necessity for a sizeable stretch of either purines or pyrimidines to be present on one strand of a duplex.

The expression of a glycosyltransferase may be also inhibited by what is referred to as “co-repression.” Co-repression refers to the phenomenon in which, when a gene having an identical or similar to the target sequence is introduced to a cell, expression of both introduced and endogenous genes becomes repressed. This phenomenon, although first observed in plant system, has been observed in certain animal systems as well. The sequence of the gene to be introduced does not have to be identical to the target sequence, but sufficient homology allows the co-repression to occur. The determination of the extent of homology depends on individual cases, and is within the ordinary skill in the art.

It would be readily apparent to one of ordinary skill in the art that other methods of gene expression inhibition that selectively target a glycosyltransferase DNA or mRNA can also be used in connection with this invention without departing from the spirit of the invention. In a specific embodiment, using techniques known to those of ordinary skill in the art, the present invention contemplates affecting the promoter region of a glycosyltransferase to effectively switch off transcription.

3. Design and Production of the RNAi Compositions

One or more of the following guidelines may be used in designing the sequence of siRNA and other nucleic acids designed to bind to a target mRNA, e.g., shRNA, stRNA, antisense oligonucleotides, ribozymes, and the like, that are advantageously used in accordance with the present invention.

Beginning with the AUG start codon of a glycosyltransferase gene, each AA dinucleotide sequence and the 3′ adjacent 16 or more nucleotides are potential siRNA targets. In a specific embodiment, the siRNA is specific for a target region that differs by at least one base pair between the wild type and mutant allele or between splice variants. In dsRNAi, the first strand is complementary to this sequence, and the other strand identical or substantially identical to the first strand. siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules may have a 3′ overhang of 2 nucleotides, such as TT. The overhanging nucleotides may be either RNA or DNA. In one embodiment, it may be desirable to choose a target region wherein the mismatch is a purine:purine mismatch.

Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website (http://www.ncbi.nih.gov). Select one or more sequences that meet the criteria for evaluation.

Another method includes selecting in the sequence of the target mRNA, a region located from about 50 to about 100 nt 3′ from the start codon. In this region, search for the following sequences: AA(N19)TT or AA(N21), where N=any nucleotide. The GC content of the selected sequence should be from about 30% to about 70%, preferably about 50%. To maximize the specificity of the RNAi, it may be desirable to use the selected sequence in a search for related sequences in the genome of interest; sequences absent from other genes are preferred. The secondary structure of the target mRNA may be determined or predicted, and it may be preferable to select a region of the mRNA that has little or no secondary structure, but it should be noted that secondary structure seems to have little impact on RNAi. When possible, sequences that bind transcription and/or translation factors should be avoided, as they might competitively inhibit the binding of a siRNA, sbRNA or stRNA (as well as other antisense oligonucleotides) to the mRNA. Further general information about the design and use of siRNA may be found in “The siRNA User Guide,” available at The Max-Planck-Institut fur Biophysikalishe Chemie website (http://www.mpibpc.mpg.de).

Negative control siRNAs should have the same nucleotide composition as the selected siRNA, but without significant sequence complementarity to the appropriate genome. Such negative controls may be designed by randomly scrambling the nucleotide sequence of the selected siRNA; a homology search can be performed to ensure that the negative control lacks homology to any other gene in the appropriate genome.

4. Delivery of Glycosyltransferase RNA Targeting Compositions

Delivery of the compositions of the present invention (e.g., siRNAs, antisense oligonucleotides, or other compositions described herein) into a patient can either be direct, e.g., the patient is directly exposed to the compositions of the present invention or compound-carrying vector, or indirect, e.g., cells are first transformed with the compositions of this invention in vitro, then transplanted into the patient for cell replacement therapy. These two approaches are known as in vivo and ex vivo therapy, respectively.

In the case of in vivo therapy, the compositions of the present invention are directly administered in vivo, where they are expressed to produce the encoded product. This can be accomplished by any of numerous methods known in the art, e.g., by constructing them as part of an appropriate nucleic acid expression vector and administering them so that they become intracellular, by infection using a defective or attenuated retroviral or other viral vector, by direct injection of naked DNA, by coating with lipids or cell-surface receptors or transfecting agents, encapsulation in liposomes, nanoparticles, microparticles, or microcapsules, by administering them in linkage to a peptide which is known to enter the cell or nucleus, or by administering them in linkage to a ligand subject to receptor-mediated endocytosis which can be used to target cell types specifically expressing the receptors. Further, the compositions of the present invention can be targeted in vivo for cell specific uptake and expression, by targeting a specific receptor. See, e.g., WO93/14188, WO 93/20221, WO 92/22635, WO92/20316, and WO 92/06180.

Ex vivo therapy involves transferring the compositions of the present invention to cells in tissue culture by methods well-known in the art such as electroporation, transfection, lipofection, microinjection, calcium phosphate mediated transfection, cell fusion, chromosome-mediated gene transfer, microcell-mediated gene transfer, spheroplast fusion, and infection with a viral vector containing the nucleic acid sequences. These techniques should provide for the stable transfer of the compositions of this invention to the cell, so that they are expressible by the cell and preferably heritable and expressible by its cell progeny. In particular embodiments, the method of transfer includes the transfer of a selectable marker to the cells. The cells are then placed under selection to isolate those cells that have taken up and are expressing the transferred compositions. The resulting recombinant cells can be delivered to a patient by various methods known in the art. Examples of the delivery methods include, but are not limited to, subcutaneous injection, skin graft, and intravenous injection.

B. Antibodies to Glycosyltransferases

The present invention contemplates the use of antibodies specific for glycosyltransferases in the treatment and prevention of cardiovascular diseases including cardiac hypertrophy. The phrases “binding specificity,” “binding specifically to, “specific binding” or otherwise any reference to an antibody to a glycosyltransferase, refers to a binding reaction that is determinative of the presence of the corresponding glycosyltransferase antigen to the antibody in a heterogeneous population of antigens and other biologics. The parameters required to achieve such specificity can be determined routinely, using conventional methods in the art including, but not limited to, competitive binding studies. The binding affinity of an antibody can also be readily determined, for example, by Scatchard analysis (Scatchard, Ann. NY Acad. Sci. 51: 660-672, 1949). In some embodiments, the immunoglobulins of the present invention bind to a glycosyltransferase at least about 5, at least about 10, at least about 100, at least about 10³, at least about 10⁴, at least 10⁵, and at least 10⁶ fold higher than to other proteins.

Various procedures known in the art may be used for the production of antibodies to a glycosyltransferase, glycosyltransferase family members or any subunit thereof, or a fragment, derivative, homolog or analog of the protein. Antibodies of the present invention include, but are not limited to, synthetic antibodies, polyclonal antibodies, monoclonal antibodies, recombinantly produced antibodies, intrabodies, multispecific antibodies (including bi-specific antibodies), human antibodies, humanized antibodies, chimeric antibodies, synthetic antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, e.g., molecules that contain an antigen binding site that immunospecifically binds to an antigen (e.g., one or more complementarity determining regions (CDRs) of an antibody).

Another embodiment for the preparation of antibodies according to the invention is the use of peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics in rational design is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting antibodies disclosed herein, but with altered and even improved characteristics. More specifically, under this rational design approach, peptide mapping may be used to determine “active” antigen recognition residues, and along with molecular modeling and molecular dynamics trajectory analysis, peptide mimic of the antibodies containing antigen contact residues from multiple CDRs may be prepared.

In some embodiments, an antibody specifically binds an epitope of the glycosyltransferase protein. It is to be understood that the peptide regions may not necessarily precisely map one epitope, but may also contain a glycosyltransferase sequence that is not immunogenic. Methods of predicting other potential epitopes to which an immunoglobulin of the invention can bind are well-known to those of skill in the art and include, without limitation, Kyte-Doolittle Analysis (Kyte, J. and Dolittle, R. F., 157 J. MOL. BIOL. 105-32 (1982)); Hopp and Woods Analysis (Hopp, T. P. and Woods, K. R., 78 PROC. NATL. ACAD. SCI. USA 3824-28 (1981); Hopp, T. J. and Woods, K. R., 20 MOL. IMMUNOL. 483-89 (1983); Hopp, T. J., 88 J. IMMUNOL. METHODS 1-18 (1986)); Jameson-Wolf Analysis (Jameson, B. A. and Wolf, H., 4 COMPUT. APPL. BIOSCI. 181-86 (1988)); and Emini Analysis (Emini et al., 140 VIROLOGY 13-20 (1985)).

Amino acid sequence variants of the antibodies of the present invention may be prepared by introducing appropriate nucleotide changes into the polynucleotide that encodes the antibody or by peptide synthesis. Such modifications include, for example, deletions from, and/or insertions into and/or substitutions of, residues within the amino acid sequences of the antibody. Any combination of deletions, insertions, and substitutions may be made to arrive at the final construct.

Amino acid sequence insertions include amino-terminal and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody of a polypeptide that increases the serum half-life of the antibody.

Another type of antibody variant is an amino acid substitution variant. These variants have at least one amino acid residue in the antibody molecule replaced by a different residue. For example, the sites of greatest interest for substitutional mutagenesis of antibodies include the hypervariable regions, but framework region (FR) alterations are also contemplated.

A useful method for the identification of certain residues or regions of the glycosyltransferase antibodies that are preferred locations for substitution, i.e., mutagenesis, is alanine scanning mutagenesis. See Cunningham & Wells, 244 SCIENCE 1081-85 (1989). Briefly, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. The amino acid locations demonstrating functional sensitivity to the substitutions are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis may be conducted at the target codon or region and the expressed antibody variants screened for the desired activity.

Substantial modifications in the biological properties of the antibody can be accomplished by selecting substitutions that differ significantly in their effect on, maintaining (i) the structure of the polypeptide backbone in the area of the substitution, for example, as a sheet or helical conformation, (ii) the charge or hydrophobicity of the molecule at the target site, or (iii) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Conservative substitutions involve exchanging of amino acids within the same class.

Any cysteine residue not involved in maintaining the proper conformation of the antibody also may be substituted, generally with serine, to improve the oxidative stability of the molecule and prevent aberrant crosslinking Conversely, cysteine bond(s) may be added to the antibody to improve its stability, particularly where the antibody is an immunoglobulin fragment such as an Fv fragment.

Another type of substitutional variant involves substituting one or more hypervariable region residues of a parent antibody. Generally, the resulting variant(s), i.e., functional equivalents as defined above, selected for further development will have improved biological properties relative to the parent antibody from which they are generated. A convenient way for generating such substitutional variants is by affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed.

In order to identify candidate hypervariable region sites for modification, alanine-scanning mutagenesis may be performed to identify hypervariable region residues contributing significantly to antigen binding. Alternatively, or additionally, it may be beneficial to analyze a crystal structure of the antibody-antigen complex to identify contact points between the antibody and antigen. Such contact residues and neighboring residues are candidates for substitution according to the techniques elaborated herein. Once generated, the panel of variants is subjected to screening as described herein and antibodies with superior properties in one or more relevant assays may be selected for further development.

It may be desirable to modify the antibodies of the present invention, i.e., create functional equivalents, with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). Caron et al., 176 J. EXP MED. 1191-95 (1992); Shopes, 148 J. IMMUNOL. 2918-22 (1992). Homodimeric antibodies with enhanced anti-tumor activity may also be prepared using heterobifunctional cross-linkers as described in Wolff et al., 53 CANCER RESEARCH 2560-65 (1993). Alternatively, an antibody can be engineered which has dual Fc regions and may thereby have enhanced complement lysis and ADCC capabilities. Stevenson et al., 3 ANTI-CANCER DRUG DESIGN 219-30 (1989).

To increase the serum half life of an antibody, one may incorporate a salvage receptor binding epitope into the antibody (especially an immunoglobulin fragment) as described in, for example, U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

Polynucleotide molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site directed) mutagenesis, PCR mutagenesis, and cassette mutagenesis of an earlier prepared variant or a non-variant version of the anti-glycosyltransferase antibodies of the present invention.

### IV. PHARMACEUTICAL COMPOSITIONS

A. Formulations

The present invention also provides pharmaceutical compositions. Such compositions comprise a glycolipid synthesis inhibitor of the present invention. The composition further comprises a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the glycolipid synthesis inhibitor is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of a glycolipid synthesis inhibitor together with a suitable amount of a pharmaceutically acceptable carrier so as to provide the form for proper administration to the patient. The formulation should suit the mode of administration.

Furthermore, a glycolipid synthesis inhibitor of the present invention can be administered with compounds that facilitate uptake of the glycolipid synthesis inhibitor by target cells or otherwise enhance transport of an inhibitor to a particular site for action. Absorption promoters, detergents and chemical irritants (e.g., keratinolytic agents) can enhance transmission of an agent into a target tissue (e.g., through the skin). For general principles regarding absorption promoters and detergents which have been used with success in mucosal delivery of organic and peptide-based drugs, see, e.g., Chien, Novel Drug Delivery Systems, Ch. 4 (Marcel Dekker, 1992). Suitable agents for use in the methods of the present invention for mucosal/nasal delivery are also described in Chang, et al., Nasal Drug Delivery, “Treatise on Controlled Drug Delivery”, Ch. 9 and Tables 3-4B thereof, (Marcel Dekker, 1992). Suitable agents which are known to enhance absorption of drugs through skin are described in Sloan, Use of Solubility Parameters from Regular Solution Theory to Describe Partitioning-Driven Processes, Ch. 5, “Prodrugs: Topical and Ocular Drug Delivery” (Marcel Dekker, 1992), and at places elsewhere in the text. All of these references are incorporated herein for the sole purpose of illustrating the level of knowledge and skill in the art concerning drug delivery techniques.

In other embodiments, a colloidal dispersion system may be used for targeted delivery of the glycolipid synthesis inhibitor to specific issue. Colloidal dispersion systems include macromolecule complexes, nanocapsules, microspheres, beads, and lipid-based systems including oil-in-water emulsions, micelles, mixed micelles, and liposomes.

B. Routes of Administration

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means.

C. Dosage Determinations

In general, the pharmaceutical compositions disclosed herein may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., a pharmaceutical composition comprising a glycolipid synthesis inhibitor in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical composition and various therapeutic agents may be independently optimized and combined to achieve a synergistic result wherein the pathology is reduced more than it would be if either were used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give by an intravenous route in an amount of about 0.0004 μg-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of humans or animals can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

D. Combination Therapy

It would be readily apparent to one of ordinary skill in the art that the pharmaceutical compositions of the present invention (e.g., the glycolipid synthesis inhibitors) can be combined with one or more therapeutic agents. In particular, the compositions of the present invention and other therapeutic agents can be administered simultaneously or sequentially by the same or different routes of administration. The determination of the identity and amount of therapeutic agent(s) for use in the methods of the present invention can be readily made by ordinarily skilled medical practitioners using standard techniques known in the art. In specific embodiments, a glycolipid synthesis inhibitor of the present invention can be administered in combination with an effective amount of a therapeutic agent that treats cardiac hypertrophy and/or any heart disease associated with cardiac hypertrophy.

Therapeutic agents include, but are not limited to, beta blockers, anti-hypertensives, cardiotonics, anti-thrombotics, vasodilators, hormone antagonists, inotropes, diuretics, endothelin antagonists, calcium channel blockers, phosphodiesterase inhibitors, ACE inhibitors, angiotensin type 2 antagonists and cytokine blockers/inhibitors, and HDAC inhibitors.

More specifically, a glycolipid synthesis inhibitor may be combined with another therapeutic agent including, but not limited to, an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof

In specific embodiments, a glycolipid synthesis inhibitor may be combined with an antihyperlipoproteinemic agent including, but not limited to, aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof, acifran, azacosterol, benfluorex, β-benzalbutyramide, camitine, chondroitin sulfate, clomestrone, detaxtran, dextran sulfate sodium, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

A glycolipid synthesis inhibitor may be combined with an antiarteriosclerotic agent such as pyridinol carbamate. In other embodiments, a glycolipid synthesis inhibitor may be combined with an antithrombotic/fibrinolytic agent including, but not limited to anticoagulants (acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin); anticoagulant antagonists, antiplatelet agents (aspirin, a dextran, dipyridamole (persantin), heparin, sulfinpyranone (anturane) and ticlopidine (ticlid)); thrombolytic agents (tissue plaminogen activator (activase), plasmin, pro-urokinase, urokinase (abbokinase) streptokinase (streptase), anistreplase/APSAC (eminase)); thrombolytic agent antagonists or combinations thereof);

In other embodiments, a glycolipid synthesis inhibitor may be combined with a blood coagulant including, but not limited to, thrombolytic agent antagonists (amiocaproic acid (amicar) and tranexamic acid (amstat); antithrombotics (anagrelide, argatroban, cilstazol, daltroban, defibrotide, enoxaparin, fraxiparine, indobufen, lamoparan, ozagrel, picotamide, plafibride, tedelparin, ticlopidine and triflusal); and anticoagulant antagonists (protamine and vitamine K1).

Alternatively, a glycolipid synthesis inhibitor may be combined with an antiarrhythmic agent including, but not limited to, Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

Non-limiting examples of sodium channel blockers include Class IA (disppyramide (norpace), procainamide (pronestyl) and quinidine (quinidex)); Class IB (lidocaine (xylocalne), tocamide (tonocard) and mexiletine (mexitil)); and Class IC antiarrhythmic agents. (encamide (enkaid) and flecamide (tambocor)).

Non-limiting examples of a beta blocker (also known as a β-adrenergic blocker, a β-adrenergic antagonist or a Class II antiarrhythmic agent) include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

Non-limiting examples of an agent that prolongs repolarization, also known as a Class III antiarrhythmic agent, include amiodarone (cordarone) and sotalol (betapace).

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexyline. In certain embodiments a calcium channel blocker comprises a long-acting dihydropyridine (nifedipine-type) calcium antagonist.

Non-limiting examples of miscellaneous antiarrhymic agents include adenosine (adenocard), digoxin (lanoxin), acecamide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.

In other embodiments, a glycolipid synthesis inhibitor may be combined with an antihypertensive agent including, but not limited to, alpha/beta blockers (labetalol (normodyne, trandate)), alpha blockers, anti-angiotensin II agents, sympatholytics, beta blockers, calcium channel blockers, vasodilators and miscellaneous antihypertensives.

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

Non-limiting examples of anti-angiotension II agents include angiotensin converting enzyme inhibitors and angiotension II receptor antagonists. Non-limiting examples of angiotensin converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

Non-limiting examples of a sympatholytic include a centrally acting sympatholytic or a peripherially acting sympatholytic. Non-limiting examples of a centrally acting sympatholytic, also known as a central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or an al-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alphal-adrenergic blocker include prazosin (minipress), doxazocin (cardura) and terazosin (hytrin).

In certain embodiments an antihypertensive agent may comprise a vasodilator (e.g., a cerebral vasodilator, a coronary vasodilator or a peripheral vasodilator). In particular embodiments, a vasodilator comprises a coronary vasodilator including, but not limited to, amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexyline, pimethylline, trapidil, tricromyl, trimetazidine, troInitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative (amosulalol, bufuralol, dilevalol, labetalol, pronethalol, sotalol and sulfinalol); a benzothiadiazine derivative (althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlonnethiazide); a N-carboxyalkyl(peptide/lactam) derivative (alacepril, captopril, cilazapril, delapril, enalapril, enalaprilat, fosinopril, lisinopril, moveltipril, perindopril, quinapril and ramipril); a dihydropyridine derivative (amlodipine, felodipine, isradipine, nicardipine, nifedipine, nilvadipine, nisoldipine and nitrendipine); a guanidine derivative (bethanidine, debrisoquin, guanabenz, guanacline, guanadrel, guanazodine, guanethidine, guanfacine, guanochlor, guanoxabenz and guanoxan); a hydrazines/phthalazine (budralazine, cadralazine, dihydralazine, endralazine, hydracarbazine, hydralazine, pheniprazine, pildralazine and todralazine); an imidazole derivative (clonidine, lofexidine, phentolamine, tiamenidine and tolonidine); a quanternary ammonium compound (azamethonium bromide, chlorisondamine chloride, hexamethonium, pentacynium bis(methylsulfate), pentamethonium bromide, pentolinium tartrate, phenactropinium chloride and trimethidinium methosulfate); a reserpine derivative (bietaserpine, deserpidine, rescinnamine, reserpine and syrosingopine); or a suflonamide derivative (ambuside, clopamide, farosemide, indapamide, quinethazone, tripamide and xipamide).

In other embodiments, a glycolipid synthesis inhibitor may be combined with a vasopressor. Vasopressors generally are used to increase blood pressure during shock, which may occur during a surgical procedure. Non-limiting examples of a vasopressor, also known as an antihypotensive include amezinium methyl sulfate, angiotensin amide, dimetofrine, dopamine, etifelmin, etilefrin, gepefrine, metaraminol, midodrine, norepinephrine, pholedrine and synephrine.

A glycolipid synthesis inhibitor may be combined with treatment agents for congestive heart failure including, but not limited to, anti-angiotension II agents, afterload-preload reduction treatment (hydralazine (apresoline) and isosorbide dinitrate (isordil, sorbitrate)), diuretics, and inotropic agents.

Non-limiting examples of a diuretic include a thiazide or benzothiadiazine derivative (e.g., althiazide, bendroflumethazide, beizthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urea.

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for hypertrophy, vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

Alternatively, therapeutic agents that can be administered in combination therapy with one or more glycolipid synthesis inhibitors include, but are not limited to, anti-inflammatory, anti-viral, anti-fungal, anti-mycobacterial, antibiotic, amoebicidal, trichomonocidal, analgesic, anti-neoplastic, anti-hypertensives, anti-microbial and/or steroid drugs, to treat cardiac hypertrophy and/or any heart disease associated with cardiac hypertrophy. In some embodiments, patients are treated with a glycolipid synthesis inhibitor in combination with one or more of the following; β-lactam antibiotics, tetracyclines, chloramphenicol, neomycin, gramicidin, bacitracin, sulfonamides, nitrofurazone, nalidixic acid, cortisone, hydrocortisone, betamethasone, dexamethasone, fluocortolone, prednisolone, triamcinolone, indomethacin, sulindac, acyclovir, amantadine, rimantadine, recombinant soluble CD4 (rsCD4), anti-receptor antibodies (e.g., for rhinoviruses), nevirapine, cidofovir (Vistide™), trisodium phosphonoformate (Foscarnet™), famcyclovir, pencyclovir, valacyclovir, nucleic acid/replication inhibitors, interferon, zidovudine (AZT, Retrovir™), didanosine (dideoxyinosine, ddI, Videx™), stavudine (d4T, Zerit™), zalcitabine (dideoxycytosine, ddC, Hivid™), nevirapine (Viramune™), lamivudine (Epivir™, 3TC), pro tease inhibitors, saquinavir (Invirase™, Fortovase™), ritonavir (Norvir™), nelfinavir (Viracept™), efavirenz (Sustiva™), abacavir (Ziagent™), amprenavir (Agenerase™) indinavir (Crixivan™), ganciclovir, AzDU, delavirdine (Kescriptor™), kaletra, trizivir, rifampin, clathiromycin, erythropoietin, colony stimulating factors (G-CSF and GM-CSF), non-nucleoside reverse transcriptase inhibitors, nucleoside inhibitors, adriamycin, fluorouracil, methotrexate, asparagyinase and combinations foregoing.

In another aspect, the glycolipid synthesis inhibitors of the present invention may be combined with other therapeutic agents including, but not limited to, immunomodulatory agents, anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), and leukotreine antagonists (e.g., montelukast, methyl xanthines, zafirlukast, and zileuton), J32-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), other anti-viral agents, and antibiotics (e.g., dactinomycin (formerly actinomycin), bleomycin, erythomycin, penicillin, mithramycin, and anthramycin (AMC)).

In various embodiments, a glycolipid synthesis inhibitor of the present invention in combination with a second therapeutic agent may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, a glycolipid synthesis inhibitor of the present invention and one or more other therapies are cyclically administered. Cycling therapy involves the administration of a first therapy (e.g., a glycolipid synthesis inhibitor) for a period of time, followed by the administration of a second therapy (e.g. a second glycolipid synthesis inhibitor or another therapeutic agent) for a period of time, optionally, followed by the administration of a third therapy for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

### V. BIOMARKERS

In one aspect of the present invention, a glycosyltransferase may be used as a biomarker. In certain embodiments, glycosyltransferases are used as a biomarker for cardiac hypertrophy. In other embodiments, glycosyltransferases are used as biomarkers for fibrosis, proliferation angiogenesis, apoptosis, atherosclerosis, and aortic intima media thickening. In further embodiments, glycosyltransferases may be used as a biomarker for cardiovascular disease.

A. Detection by Mass Spectrometry

In one aspect, the biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like.

In particular embodiments, the biomarkers of the present invention are detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g. 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic coelution of multiple transitions for a given analyte. The terms SRM/MRM are occasionally used also to describe experiments conducted in mass spectrometers other than triple quadrupoles (e.g. in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application the terms SRM and MRM or also SRM/MRM can be used interchangeably, since they both refer to the same mass spectrometer operating principle. As a matter of clarity, the term SRM is used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissocation (also known as CID or collision-induced dissociation), HCD (higher energy CID), ECD (electron capture dissociation), PD (photodissociation) or ETD (electron transfer dissociation).

In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein. In another embodiment, the mass spectrometric technique is multiple reaction monitoring (MRM) or quantitative MRM.

In an alternative embodiment, the mass spectrometric technique comprises surface enhanced laser desorption and ionization or “SELDI,” as described, for example, in U.S. Pat. No. 6,225,047 and No. 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

B. Detection by Immunoassay

In another embodiment, the biomarkers of the present invention can be measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

The present invention contemplates traditional immunoassays including, for example, sandwich immunoassays including ELISA or fluorescence-based immunoassays, as well as other enzyme immunoassays. Nephelometry is an assay performed in liquid phase, in which antibodies are in solution. Binding of the antigen to the antibody results in changes in absorbance, which is measured. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry. The Quantikine immunoassay developed by R&D Systems, Inc. (Minneapolis, Minn.) may also be used in the methods of the present invention.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds glycosyltransferase, atrial natriuretic peptide (ANP), etc. and/or one or more of their breakdown products might be used. Aptamers are nucleic acid-based molecules that bind specific ligands. Methods for making aptamers with a particular binding specificity are known as detailed in U.S. Pat. No. 5,475,096; No. 5,670,637; No. 5,696,249; No. 5,270,163; No. 5,707,796; No. 5,595,877; No. 5,660,985; No. 5,567,588; No. 5,683,867; No. 5,637,459; and No. 6,011,020.

C. Detection by Electrochemicaluminescent Assay

In several embodiments, the glycosyltransferase biomarkers and other biomarkers may be detected by means of an electrochemicaluminescent assay developed by Meso Scale Discovery (Gaithersrburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. No. 7,497,997; No. 7,491,540; No. 7,288,410; No. 7,036,946; No. 7,052,861; No. 6,977,722; No. 6,919,173; No. 6,673,533; No. 6,413,783; No. 6,362,011; No. 6,319,670; No. 6,207,369; No. 6,140,045; No. 6,090,545; and No. 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534. In specific embodiments, novel glycosyltransferase proteins/isoforms can be quantified using Mesoscale technology.

D. Other Methods for Detecting Biomarkers

The biomarkers of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication No. WO 00/56934; and PCT International Publication No. WO 03/048768.

D. Sample Preparation

In several embodiments of the present invention, a blood sample is tested for the presence or absence of one or more biomarkers including a glycosyltransferase. The step of collecting a sample such as a blood sample from a subject can be carried out by phlebotomy or any other suitable technique. The blood sample may be further processed to provide a serum sample or other suitable blood fraction, such as plasma.

In alternative embodiments of the present invention, a tissue sample may be taken and tested for the presence or absence of one or more biomarkers including a glycosyltransferase. Tissue or cell samples can be removed from almost any part of the body. The most appropriate method for obtaining a tissue sample depends on the type of disease or condition that is suspected or diagnosed. In particular, biopsy methods include needle (e.g. fine needle aspiration), endoscopic, and excisional. Variations of these methods and the necessary devices used in such methods are known to those of ordinary skill in the art.

### VI. DETERMINATION OF SUBJECT CARDIAC HYPERTROPHY STATUS

A. Glycosyltransferase Biomarker

The biomarkers of the present invention can be used in diagnostic tests to assess cardiac hypertrophy status in a subject. The phrase “cardiac hypertrophy status” includes any distinguishable manifestation of the condition, including non-condition. For example, status includes, without limitation, the presence or absence of condition (e.g., cardiac hypertrophy v. non-cardiac hypertrophy), the risk of developing the condition, the stage of the condition, the progress of condition (e.g., progress of condition or remission of the condition over time) and the effectiveness or response to treatment of the condition. Based on this status, further procedures may be indicated, including additional diagnostic tests or therapeutic procedures or regimens. For ease of reference, although the glycosyltransferase biomarker is useful in the treatment of cardiac hypertrophy, fibrosis, proliferation angiogenesis, apoptosis, atherosclerosis, aortic intima media thickening, and potentially cardiovascular disease, it may be referred to specifically as being useful in the treatment of cardiac hypertrophy. A reference to the use of the glycosyltransferase biomarker in cardiac hypertrophy shall be understood to mean cardiac hypertrophy, fibrosis, proliferation angiogenesis, apoptosis, atherosclerosis, aortic intima media thickening and potentially cardiovascular disease (e.g., atherosclerosis) as well.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the glycosyltransferase biomarkers of the present invention may show a statistical difference in different cardiac hypertrophy statuses of at least p<0.05, p<10⁻², p<10⁻³, p<10⁻⁴ or p<10⁻⁵. Diagnostic tests that use this biomarker alone or in combination with other known biomarkers may show a sensitivity and specificity of at least 75%, at least 80%, at least 85%, at least 90%, at least 95%, at least 98% and about 100%. In a specific embodiment, the sensitivity and specificity is at least 68%.

The glycosyltransferase biomarkers are differentially present in cardiac hypertrophy, and, therefore, are useful in aiding in the determination of cardiac hypertrophy status. In specific embodiments, the biomarker is measured in a subject sample using the methods described herein. The measurement may then be compared with a diagnostic amount or cut-off that distinguishes a positive cardiac hypertrophy status from a negative cardiac hypertrophy status. The diagnostic amount represents a measured amount of a biomarker above which or below which a subject is classified as having a particular cardiac hypertrophy status. For example, if the biomarker is up-regulated compared to normal during cardiac hypertrophy, then a measured amount above the diagnostic cutoff provides a diagnosis of cardiac hypertrophy. Alternatively, if the biomarker is down-regulated during cardiac hypertrophy, then a measured amount below the diagnostic cutoff provides a diagnosis of cardiac hypertrophy. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different cardiac hypertrophy statuses, and drawing the cut-off to suit the desired levels of specificity and sensitivity.

B. Biomarker Panels Including Glycosyltransferases

As the skilled artisan will appreciate there are many ways to use the measurements of two or more markers in order to improve the diagnostic question under investigation. In a quite simple, but nonetheless often effective approach, a positive result is assumed if a sample is positive for at least one of the markers investigated.

Frequently, however, the combination of markers is evaluated. Preferably the values measured for markers of a marker panel are mathematically combined and the combined value is correlated to the underlying diagnostic question. Biomarker values may be combined by any appropriate state of the art mathematical method. Well-known mathematical methods for correlating a marker combination to a disease employ methods like discriminant analysis (DA) (e.g., linear-, quadratic-, regularized-DA), Kernel Methods (e.g., SVM), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in correlating biomarker combination of the present invention e.g. to the absence or presence of cardiac hypertrophy is selected from DA (e.g., Linear-, Quadratic-, Regularized Discriminant Analysis), Kernel Methods (e.g., SVM), Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression). Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

B. Determining Risk of Developing Disease

In a specific embodiment, the present invention provides methods for determining the risk of developing cardiac hypertrophy in a subject. Biomarker amounts or patterns are characteristic of various risk states, e.g., high, medium or low. The risk of developing a disease is determined by measuring the relevant biomarker or biomarkers and then either submitting them to a classification algorithm or comparing them with a reference amount and/or pattern of biomarkers that is associated with the particular risk level.

C. Determining Stage of Disease

In another embodiment, the present invention provides methods for determining the stage of cardiac hypertrophy in a subject. Each stage of the disease has a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage.

D. Determining Course of Disease

In one embodiment, the present invention provides methods for determining the course of cardiac hypertrophy in a subject. Disease course refers to changes in disease status over time, including disease progression (worsening) and disease regression (improvement). Over time, the amounts or relative amounts (e.g., the pattern) of the biomarker(s) changes. For example, biomarker glycosyltransferase is increased with cardiac hypertrophy, while biomarker “X” may be decreased in cardiac hypertrophy. Therefore, the trend of these biomarkers, either increased or decreased over time toward diseased or non-diseased indicates the course of the condition. Accordingly, this method involves measuring one or more biomarkers in a subject at least two different time points, e.g., a first time and a second time, and comparing the change in amounts, if any. The course of disease is determined based on these comparisons.

E. Subject Management

In certain embodiments of the methods of qualifying cardiac hypertrophy status, the methods further comprise managing subject treatment based on the status. Such management includes the actions of the physician or clinician subsequent to determining cardiac hypertrophy status. For example, if a physician makes a diagnosis of cardiac hypertrophy, then a certain regime of treatment, such as prescription or administration of therapeutic agent might follow. Alternatively, a diagnosis of non-cardiac hypertrophy might be followed with further testing to determine a specific disease that the patient might be suffering from. Also, further tests may be called for if the diagnostic test gives an inconclusive result on cardiac hypertrophy status.

F. Determining Therapeutic Efficacy of Pharmaceutical Drug

In another embodiment, the present invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of one or more of the biomarkers of the present invention may change toward a non-disease profile. Therefore, one can follow the course of the amounts of one or more biomarkers in the subject during the course of treatment. Accordingly, this method involves measuring one or more biomarkers (including a glycosyltransferase) in a subject receiving drug therapy, and correlating the amounts of the biomarkers with the disease status of the subject. One embodiment of this method involves determining the levels of one or more biomarkers at least two different time points during a course of drug therapy, e.g., a first time and a second time, and comparing the change in amounts of the biomarkers, if any. For example, the one or more biomarkers can be measured before and after drug administration or at two different time points during drug administration. The effect of therapy is determined based on these comparisons. If a treatment is effective, then one or more biomarkers will trend toward normal, while if treatment is ineffective, the one or more biomarkers will trend toward disease indications. If a treatment is effective, then the one or more biomarkers will trend toward normal, while if treatment is ineffective, the one or more biomarkers will trend toward disease indications.

G. Generation of Classification Algorithms for Qualifying Disease Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART-classification and regression trees), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”), U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers. The classification algorithms, in turn, form the base for diagnostic tests by providing diagnostic values (e.g., cut-off points) for biomarkers used singly or in combination.

H. Kits for the Detection of Cardiac Hypertrophy Biomarkers

In another aspect, the present invention provides kits for qualifying cardiac hypertrophy status, which kits are used to detect the glycosyltransferase biomarkers and optionally other biomarkers. The kits may also be used to detect glycosyltransferase biomarkers to diagnose fibrosis, proliferation angiogenesis, apoptosis, atherosclerosis, aortic intima media thickening, and potentially cardiovascular disease. In a specific embodiment, the kit is provided as an ELISA kit comprising an antibody to one or more glycosyltransferases. The ELISA kit may comprise a solid support, such as a chip, microtiter plate (e.g., a 96-well plate), bead, or resin having a glycosyltransferase capture reagent attached thereon. The kit may further comprise a means for detecting one or more glycosyltransferases, such as an anti-glycosyltransferase antibodies, and a secondary antibody-signal complex such as horseradish peroxidase (HRP)-conjugated goat anti-rabbit IgG antibody and tetramethyl benzidine (TMB) as a substrate for HRP.

The kit for qualifying cardiac hypertrophy status may be provided as an immuno-chromatography strip comprising a membrane on which a glycosyltransferase antibody is immobilized, and a means for detecting a glycosyltransferase, e.g., a gold particle bound glycosyltransferase antibody, where the membrane, includes NC membrane and PVDF membrane. The kit may comprise a plastic plate on which a sample application pad, a gold particle bound glycosyltransferase antibody temporally immobilized on a glass fiber filter, a nitrocellulose membrane on which a glycosyltransferase antibody band and a secondary antibody band are immobilized and an absorbent pad are positioned in a serial manner, so as to keep continuous capillary flow of blood serum.

A patient can be diagnosed by adding blood or blood serum from the patient to the kit and detecting a glycosyltransferase conjugated with a glycosyltransferase antibody, specifically, by a method which comprises the steps of: (i) collecting blood or blood serum from the patient; (ii) separating blood serum from the patient's blood; (iii) adding the blood serum from patient to a diagnostic kit; and, (iv) detecting a glycosyltransferase conjugated with a glycosyltransferase antibody. In this method, the glycosyltransferase antibodies are brought into contact with the patient's blood. If glycosyltransferase is present in the sample, the glycosyltransferase antibodies will bind to the sample, or a portion thereof. In other kit and diagnostic embodiments, blood or blood serum need not be collected from the patient (i.e., it is already collected). Moreover, in other embodiments, the sample may comprise a tissue biopsy sample.

The kit can also comprise a washing solution or instructions for making a washing solution, in which the combination of the capture reagent and the washing solution allows capture of the biomarker or biomarkers on the solid support for subsequent detection by, e.g., an antibody or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected. In yet another embodiment, the kit can comprise one or more containers with biomarker samples, to be used as standard(s) for calibration.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### To Determine the Efficacy of D-PDMP in Preventing Atherosclerosis and Cardiac Hypertrophy in apoE−/− Mice

A high fat (2%) and cholesterol (1.2%) diet (Research diet) was fed to apoE−/− mice, w/o D-PDMP (5,10 mpk) for 12, 20, and 36 weeks. As described below, the hyperlipidemic diet in Apo E−/− mice significantly increased vascular wall thickening, accumulation of atherosclerotic plaque along the bifurcation of the aortic branch and aortic root area, decreased cardiac contractility, increased left ventricle hypertrophy (LVH) and fibrosis and showed a marked increase in a PWV independent of blood pressure. Interestingly, mice treated with 10 mpk of D-PDMP exhibited significantly clear arterial wall area, decrease in cardiovascular wall thickening and fibrosis and improved cardiac contractility and reduces arterial stiffness (PWV) compared to mice treated with 5 mpk of D-PDMP and placebo. Measurement of several glycolipid glycotransferases showed that feeding a hyperlipidemic diet increased the activity of glucosylceramide synthase and lactosylceramide synthase in the artery wall of Apo E−/− mice. This was completely prevented by feeding D-PDMP. Thus, D-PDMP is therapeutically an anti-atherogenic and cardioprotective treatment with concomitant reduction of arterial stiffness.

### Materials and Methods

Ultrasound (US) Imaging.

Real time, in vivo ultrasonic imaging (VEVO 2100, Visualsonic, Toronto, Canada) system equipped with 40 MHz probe was used. Data were acquired in 2-modes, M-mode and color flow data. Using left ventricular end diastolic dimension (LVEDD) and left ventricular end systolic dimension (LVESD), the percent left ventricular contractility (Fractional Shortening, FS %) was derived based on the following equation: FS (%)=[(LVEDD−LVESD)/LVEDD]×100. Left ventricular mass (LVmass) was derived and used in the assessment of left ventricular hypertrophy and enlargement, using the following equation: LVmass (mg): 1.055 [(IVSD+LVEDD+PWTED)³−(LVEDD)³], where: 1.055 is the specific gravity of the myocardium.

PWV and Blood Pressure.

Aortic pulse wave velocity (PWV) was measured with mice placed supine under anesthesia with 1.5% isoflurane. An ECG-triggered, 2-mm, 10-MHz Doppler probe was used to measure blood flow at the thoracic and abdominal aorta. PWV was calculated by the thoracic-abdominal distance divided by the pulse transit time between the thoracic and abdominal measurement sites. Blood pressure was acquired using the CODA tail cuff system (Kent Scientific Co, USA) to measure systolic and diastolic pressure. Estimated the effect of mean pressure (MP(mmHg) on PWV (m/sec) was calculated using the following equation: PWVc=0.0006(MP)²-0.0564MP+3.9711.

Glycosyltransferase and Glycosylhydrolase Activity Assays.

Lactosylceramide synthase activity in aortic preparations was measured using a protocol detailed previously [11] with the following modifications. Briefly, flash frozen aortic tissue was homogenized in Tris-HCl buffer (pH 7.4) containing Triton-X-100 and centrifuged at 10,000 rpm for 15 min. The protein mass in the supernatant was measured; 100 μm of sample protein was used in enzymatic assays in triplicate. 14C-UDP-galactose (American Radiolabel Company, St Louis, Mo.) served as the donor of galactose. Glucosylceramide (Matreya Chemicals, PA) served as the acceptor in this assay. 14C-LacCer generated from this assay was quantified by scintillation spectrophotometry. Glucosylceramide synthase activity was measured using ceramide as the acceptor and [3H] UDP-glucose as the glucose donor. Glucosylceramide hydrolase activity was measured using [3H] glucosylceramide (American Radiolabel Company), as the substrate according to previously published protocols.

Atrial Natriuretic Peptide (ANP) RNA Expression Level in Left Ventricular Tissues.

RNA was extracted from ventricular tissues of Apo E−/−, vehicle, 5 mpk and 10 mpk treated mice and RT PCR analysis was performed using the gene specific primer for Atrial natriuretic peptide (ANP). For RT-PCR reactions (20 μl), equal amount (500 ng) of total RNA was used. Expression levels of GAPDH was used to normalize for variations in the amount of RNA. RNA expression levels were quantified by using ImageJ v.1.45s (NIH, USA) software. ANT mass is increased in transgenic mice (apo E−/−) mice fed a high fat and cholesterol diet (data not shown).

Histology.

H&E, Masson's trichrome stained aorta and heart tissue was used for histological analysis for cardiovascular wall thickening and plaque accumulation.

Statistical Analysis.

One way analysis of variance and the Bonferonni's Multiple Comparisons Test with p<0.05 was considered significant. Mean and standard error (Mean±SEM) was used.

### Results

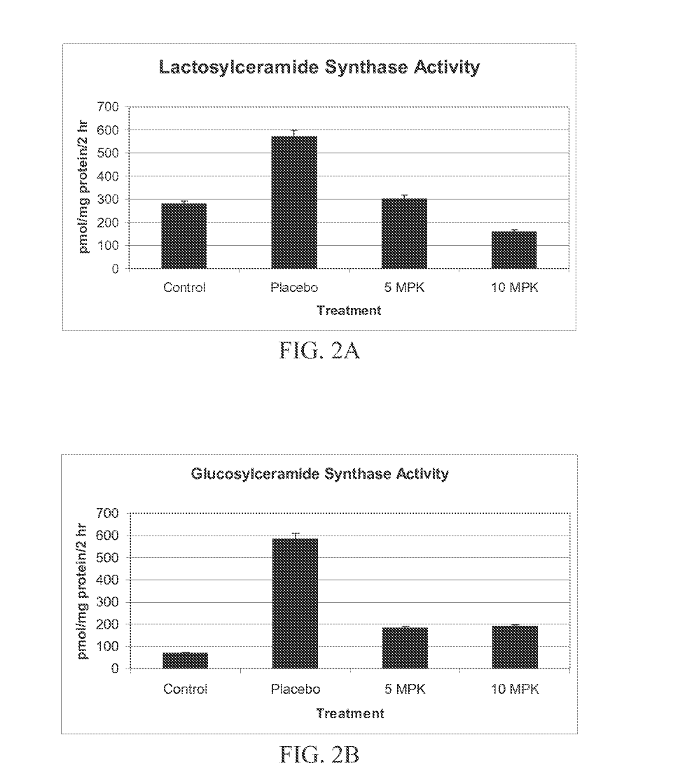

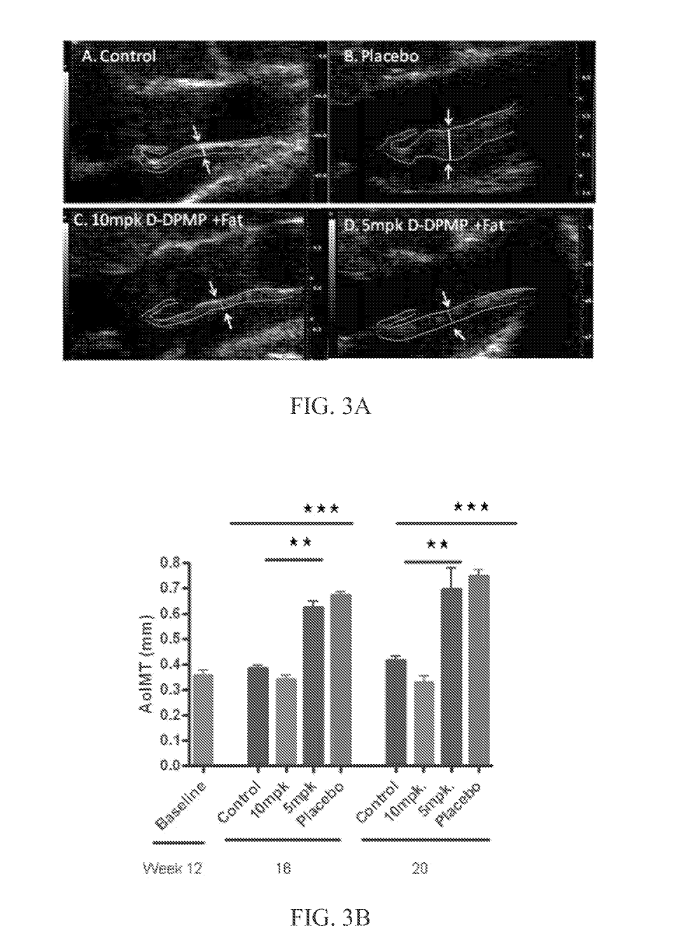

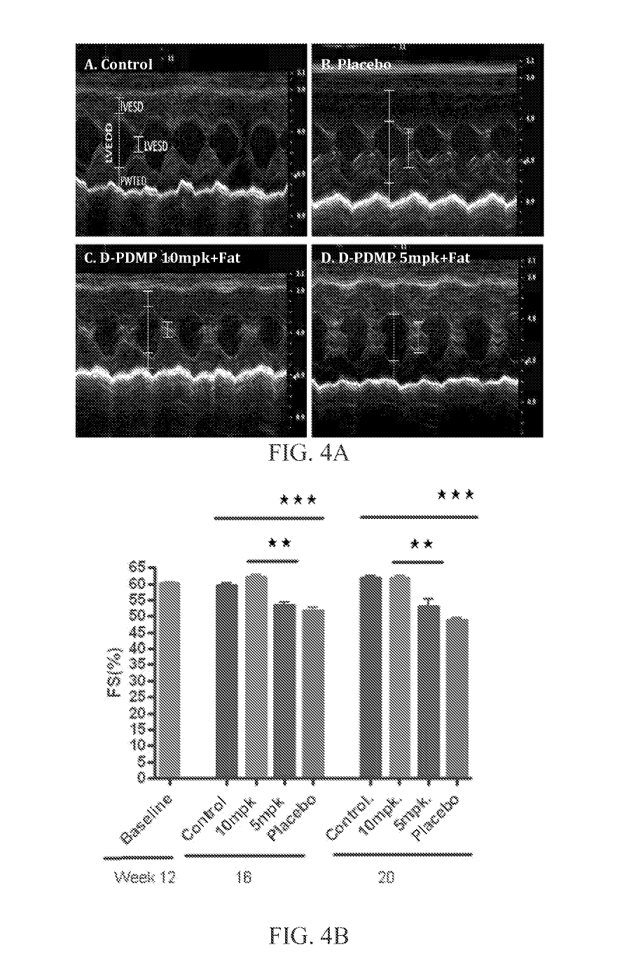

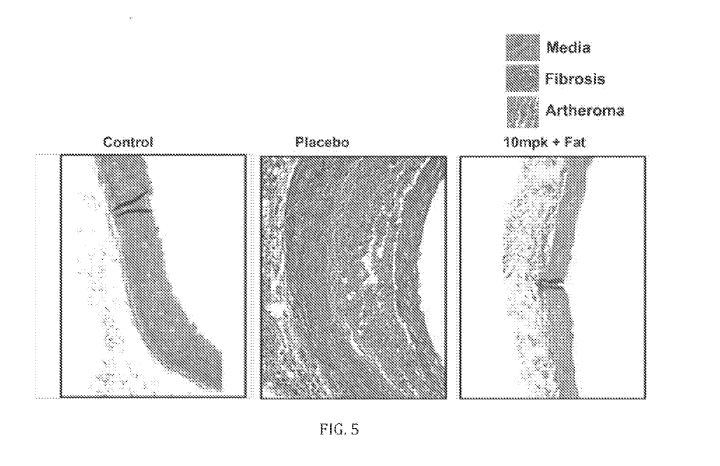

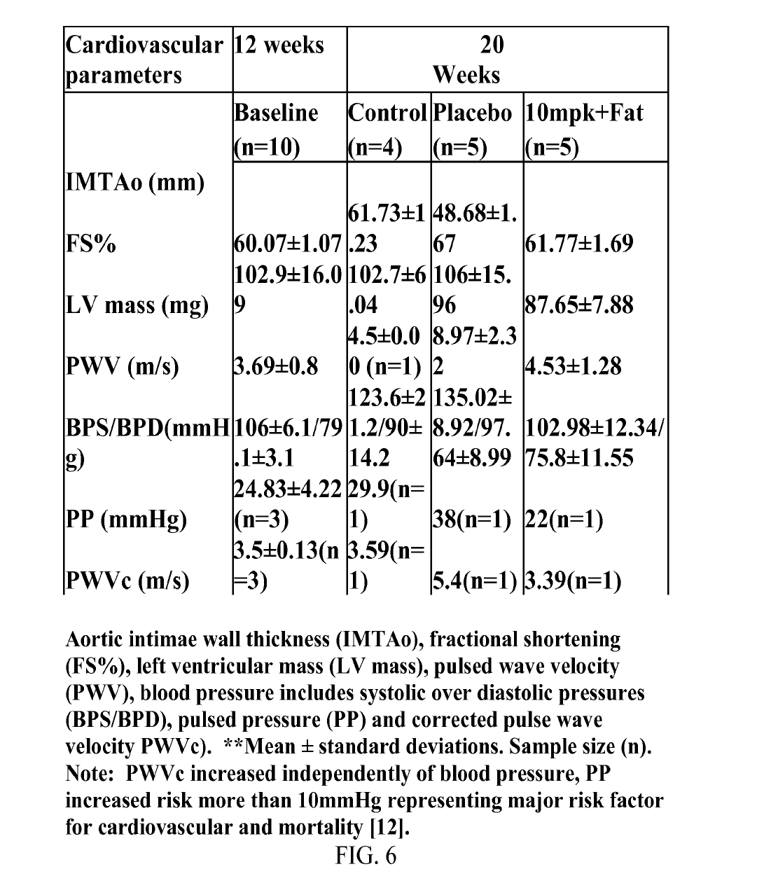

It was observed that feeding a hyperlipidemic diet increases the activity of LCS and glucosylceramide synthase activity ˜2-5 fold, respectively. D-PDMP dose-dependently decreased LCS activity in the order of ˜52% and 32%, respectively upon feeding 5 and 10 mpk drug as compared to hyperlipidemic mouse aorta (FIG. 2A). The activity of glucosylceramide synthase was also decreased upon treatment with D-PDMP, but this was not dose-dependent (FIG. 2B). The ultrasound studies revealed a marked increase in aortic intima media thickening (IMT) (FIG. 3A, B) in mice fed a hyperlipidemic diet (placebo) compared to control mice fed regular mice chow. This was reversed by feeding D-PDMP. The M mode ultra sound of the heart revealed significant hypertrophy and dysfunction in mice fed a hyperlipidemic diet (FIG. 4A, B) compared to control (FIG. 4A, Panel A) and complete protection upon treatment with D-PDMP in a dose dependent manner (FIG. 4C, D). Feeding a hyperlipidemic diet markedly increased systolic and diastolic blood pressure in mice. This was prevented by the use of the glycolipid synthesis inhibitor (Table 1).

TABLE 1
Cardiovascular Parameters in 12 and 20 Week
Old Mice Fed a Hyperlipidemic Diet with and
without Glycolipid Synthesis Inhibitor

| | Blood Pressure* (mmHg) | | Fractional Shortening (%) | |
|---|---|---|---|---|
| Treatment | 12 Weeks | 20 Weeks | 12 Weeks | 20 Weeks |
| Control | **106 ± 6.1 | 123.6 ± 21.2 | 60.07 ± 1.07 | 61.73 ± 1.23 |
| (n = 4) | 79.1 ± 3.1 | 90 ± 14.2 | | |
| Placebo | | 135.02 ± 8.92 | | 48.68 ± 1.67 |
| (n = 5) | | 97.64 ± 8.99 | | |
| (Vehicle + | | | | |
| Fat) | | | | |
| 10MPK | | 102.98 ± 12.34 | | 61.77 ± 1.69 |
| D-PDMP + | | 75.8 ± 11.55 | | |
| Fat (n = 5) | | | | |

The present inventor's finding that inhibition of glycolipid synthesis prevents aortic intima thickening in a transgenic mouse model of hyperlipidemia is highly significant. An unexpected and novel finding is that a diet rich in fat and cholesterol can markedly increase artery as well as cardiac hypertrophy and dysfunction. This phenotype was mitigated by inhibiting glycolipid synthesis.

Both glycolipid glycosyltransferases activity in the aorta and ANP expression in the left ventricle were significantly increased correlating with a significantly impaired cardiac contractility (FS %), increased in LV mass and AoIMT, increased PWV as well as blood pressure in the placebo mice. These changes were prevented and/or reversed by D-PDMP treatment. Therefore, D-PDMP therapeutically is an anti-atherogenic, cardioprotective treatment with concomitant reduction of arterial stiffness and cardiac hypertrophy.

### Example 2

### Prevent of Cardiac Hypertrophy in apoE−/− Mice Fed Western Diet and C57Bl-6 Mice Subject to Trans-Aortic Constriction by Inhibiting Glycosphingolipid Synthesis

Nearly one in three persons world-wide is afflicted by high blood pressure. In response to this increase in blood volume, stress and pressure, the cardiomyocytes in the left ventricle in the heart increase Ca2+ by recruiting transient receptor potential channels and/or sodium-hydrogen exchanger-1. Both angiotensin-1 and endothelin-1 are proteins that cause vasoconstriction and thus raise blood pressure. Other studies point at extra cellular signal related kinase-1 (ERK-1)/p44 mitogen activated protein kinase (MAPK) which has been implicated to activate the sodium hydrogen exchanger-1(NHE-1). However, nothing is known about the role of glycosphingolipid glycosyltransferases and glycosphingolipids in cardiac hypertrophy.

We have previously shown that diverse growth factors, pro inflammatory cytokines such as tumor necrosis factor, and -alpha and oxidized-LDL can activate a glycosphingolipid glycosyltransferase termed lactosylceramide synthase (LCS) via phosphorylation. Chatterjee et al., 9 CURR. DRUG TARGETS 272-81 (2008); Chatterjee S., 18 ART. THR. VASO. BIOL. 1523-33 (1998). The main function of LCS is to transfer galactose from UDP-Gal to glucosylceramide to form lactosylceramide (LacCer). In turn, LacCer can activate NADPH oxidase thus generating free oxygen radicals which activate downstream signaling kinase such as ERK-1 to induce vascular cell proliferation and angiogenesis. Our findings and others have validated these signal transduction pathways in several in vitro and in vivo animal models.

The present inventors hypothesized that inhibiting LCS activity and therefore p44MAPK may well ameliorate cardiac hypertrophy. In this report, we have used two different animal models to examine the role of GSL glycosyltransferase and GSL in cardiac hypertrophy.

First we used the apoE−/−male mice fed a western diet over a period of 6 months to expedite atherosclerosis. Second, we used normal mice (C57B1-6) subjected to trans-aortic constriction which causes severe cardiac hypertrophy within weeks. Herein, we show that inhibiting the activity of GSL glycosyltransferase by the use of D-PDMP can ameliorate cardiac hypertrophy in both of these animal models.

### Materials and Methods

Treatment of apolipoproteinE−/− Mice Fed a Western Diet with a Glycosyltransferase Inhibitor.

Apoliporotein E-deficient (ApoE (−/−), male aged 11 weeks were purchased from the Jackson Laboratory, Bar Harbor, Me. At the age of 12 weeks, the Apo E(−/−) mice were started on a high fat and high cholesterol diet (HFHC) consisting of 20% fat, and 1.25% cholesterol (D12108C, Research Diet Inc., New Brunswick, N.J.) for 20 to 36 weeks with and without treatment with D-PDMP (5 mg/kg, 10 mg/kg) and compared to control fed only chow diet and placebo mice fed HFHC+Vehicle (composed of 5% Tween-80 in phosphate buffered saline). Diet was rationed once a week to estimate the weekly growth rate and food intake. Physiological studies were performed around the age of 12, 20 and 36 weeks. Blood was drawn to prepare serum and heart tissues were harvested and studied at molecular and histopathological level. Tissues were harvested at 12, 20 and 36 weeks of age.

Animals were subject to physiological measurements, e.g., weight, blood pressure, ultra sound to measure fractional shortening (FS), left ventricular mass (LVM), heart weight to tibia length ratio, heart anatomy etc. A group of mice (N=5) were euthanized to obtain base line values for aorta and blood samples were collected. The rest of the mice were divided in to several groups. These were: Placebo (mice fed vehicle only (5% Tween-80 in phosphate buffered saline), 5 mpk D-PDMP solubilized in vehicle and 10 mpk D-PDMP solubilized in vehicle). Placebo and D-PDMP was delivered daily by oral gavage. The physiological measurements were repeated at 20 weeks and 36 week interval and then the mice were euthanized by asphyxia. This protocol was approved by the committee for research on animals at the Johns Hopkins University.

Trans-Aortic Constriction (TAC) in Normal (C57B1-6) Mice.

Normal male mice (C57B1-6), 11 weeks old, were also purchased from the Jackson Laboratory, Bar Harbor, Me.). After having measured the basal physiologic parameters such as blood pressure, weight, and ultra sound measurements, the 9 mice were divided as follows. Six mice were given 100 μL of vehicle (5% Tween-80 in phosphate—buffered saline) by oral gavage. The other three mice were fed 10 mpk of D-PDMP solubilized in vehicle. This procedure was continued for 7 days. Next, 3 mice in mice fed vehicle group and 3 mice fed D-PDMP were subject to trans-aortic constriction (Cingolani et al., 109 CIRC. RES. 00-00 (2011). At one week, two week and six weeks post-surgery, the mice were subjected to M mode ultrasound analysis. Finally, blood was drawn, and the animals were euthanized. The heart was excised, sliced in halves and its anatomy photographed.

Measurement of Atherosclerotic Lesions.

Trans-thoracic echocardiography was performed in conscious mice using the 2100 Visualsonic ultrasound device (Toronto, Ontario, Canada), and the Sequoia Acuson C256 (Malvern, Pa.) system, equipped with a 40 MHz and 15 MHz linear transducer, respectively. M-mode echocardiogram was obtained in the parasternal short axes view of the left ventricle (LV) at the level of the papillary muscles and at sweep speed of 200 mm/sec. From left ventricular end diastolic (LVEDD) and end systolic dimension (LVESD), the percent left ventricular contractility or the fractional shortening (FS) and left ventricular mass (LV mass) were derived based from the following equation:

FS(%)=[(LVEDD−LVESD)/LVEDD]×100

The left ventricular mass (LV mass) was derived and used in the assessment of left ventricular hypertrophy and enlargement, using the following equation:

LV mass (mg): 1.055[(IVSD+LVEDD+PWTED)³−(LVEDD)³],

where 1.055 is the specific gravity of the myocardium, IVSD is the interventricular septal thickness at end diastole and PWTED is the posterior wall thickness at end diastole.

Chemicals and Supplies.

All chemicals were from Sigma-Aldrich (St Louis, Mo.) unless specified otherwise. D-PDMP was purchased from Matreya, LLC (Pleasant Gap, Pa.).

Histopathology.

Masson-trichrome staining of thin heart tissue sections was performed and photographed.

Measurement of Glycosphingolipid Glycosyltransferase Activity.

Approximately 10 mg of heart tissue were homogenized in Tris buffer (pH 7.8) and centrifuged at 10,000 rpm for 10 min. The supernatant was used as a source for enzyme. [³H]UDP-Gal (American Radiolabled company, St Louis, Mo.) served as the nucleotide sugar donor in LacCer synthase assay and [³H]UDP-Glc served as the nucleotide sugar donor in GlcCer synthase assay. The details of these assays have been described previously. See Chatterjee, S., 311 METHODS IN ENZYMOLOGY 73-81 (2000). All assays were conducted in triplicate from 3 to 5 aorta samples from mice in each group.

HPLC Analysis of Glycosphingolipid Levels in Heart Tissue in apoE−/− Mice and TAC Mice.

Approximately 10 mg of heart tissue was homogenized in chloroform-methanol (2:1, v/v) and lipids were extracted. Bligh et al., 37.8 CAN. J. BIOCHEM. AND PHYSIOL. 911-17 (1959). The total lipid extracts were dried in nitrogen and subject to deacylation using sphingosine ceramide N-deacylation. Followed by o-phthalaldehyde derivatization and quantification of the levels of glucosylceramide and lactosylceramide by reversed phase HPLC (RP-HPLC) (Zama et. al. 19.7 GLYCOBIOLOGY 767-75 (2009). A C18 column was used with an isocratic organic mobile phase (methanol-water, 88:12, v/v) and calibrated with standard glycosphingolipids of known chemical structure and column affinity. All samples were analyzed in triplicate and a representative quantity (n=3) of heart tissue samples was used for each treatment from control, placebo, 5 mpk and 10 mpk D-PDMP-treated apoE−/− mice.

Quantitative Real-Time PCR.

Approximately 10-mg of left ventricular tissue was homogenized from each mice and total RNA was isolated using TRIzol reagent according to the manufacturer's instruction (Invitrogen). Two micrograms of RNA was reverse-transcribed with SuperScript II (Invitrogen, USA) using random primers. Real-time PCR was performed using SYBR Green PCR Master Mix (Applied Biosystems, USA) in an Applied Biosystems Step One Real time PCR system with the following thermal cycling conditions: 10 min. at 95° C., followed by 40 cycles of 95° C. for 15 s and 60° C. for 1 min. for denaturation, annealing and elongation. Relative mRNA levels were calculated by the method of 2^{-DDCt}. Data were normalized to GAPDH mRNA levels. To determine the specificity of amplification, melting curve analysis was applied to all final PCR products. All samples were performed in triplicate. Primers were synthesized by Integrated DNA Technologies (Coralville, USA). Expression suite software (Applied Biosystems) was used to analyze the data.

Western Immunoblot Analysis of p44MAPK and p-AKT-1 in Heart Tissues.

About 10 mg of left ventricular tissue were homogenized in Trsi HCl buffer with SDS and centrifuged. About 70 μg of supernatant protein was subjected to SDS gel electrophoresis. Antibody against p-44MAPK, GAPDH and TGF-B were purchased from Cell Signaling. Following immunoblotting, the gel bands were subjected to densitometric scanning using a Bethold gel scanner.

Statistical Analysis.

All values are expressed as Mean±SEM. Comparison between groups was performed by 1-way ANOVA with the Bonferroni's multiple comparison tests. Comparisons between two groups were performed using non-paired 2-tailed Student t test. A value of P<0.05 was considered significant.

This study demonstrates the following: 1. That both feeding a western diet such as the HCHF diet to transgenic mice, e.g., apolipoprotienE−/− mice, and trans-aortic constriction in normal mice exerts a time-dependent increase in cardiac hypertrophy; 2. This in turn raises the serum levels of oxidized LDL; 3. Oxidized LDL activates lactosylceramide synthase and generates a lipid second messenger called lactosylceramide; 3. In turn, lactosylceramide phosphorylates p44MAPK and may induce the sodium hydrogen exchanger, turn on TGF-b ANP, BNP levels all considered as biomarkers in cardiac hypertrophy; and 4. Continuous treatment with D-PDMP in the HCHF fed apoE−/− mice and pre-treatment with D-PDMP before TAC can mitigate this phenotype.

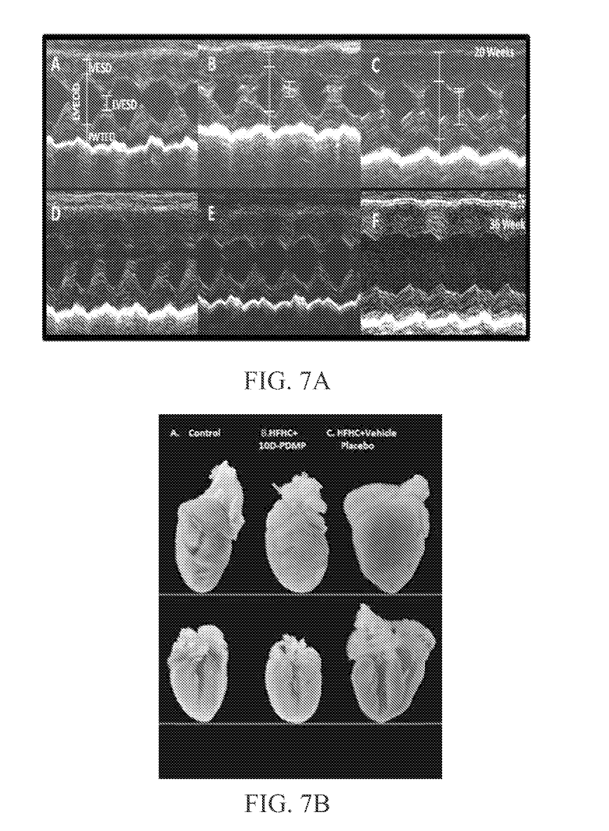

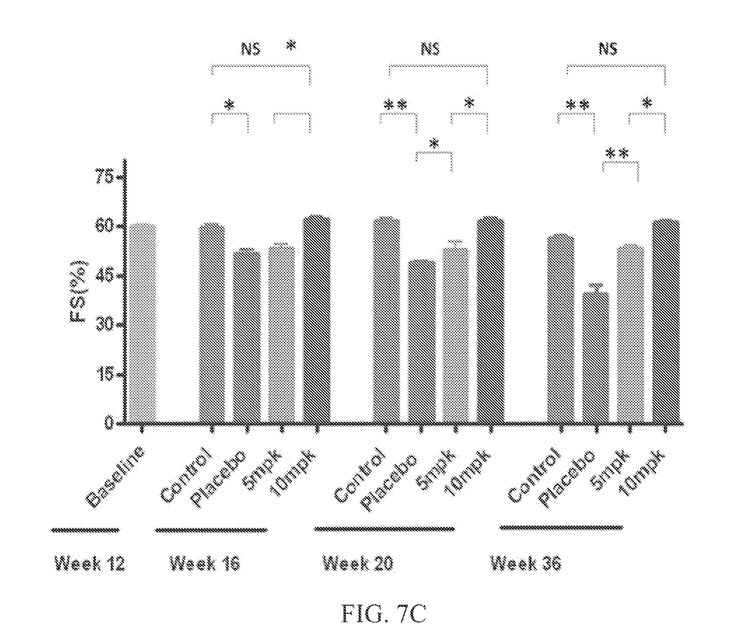

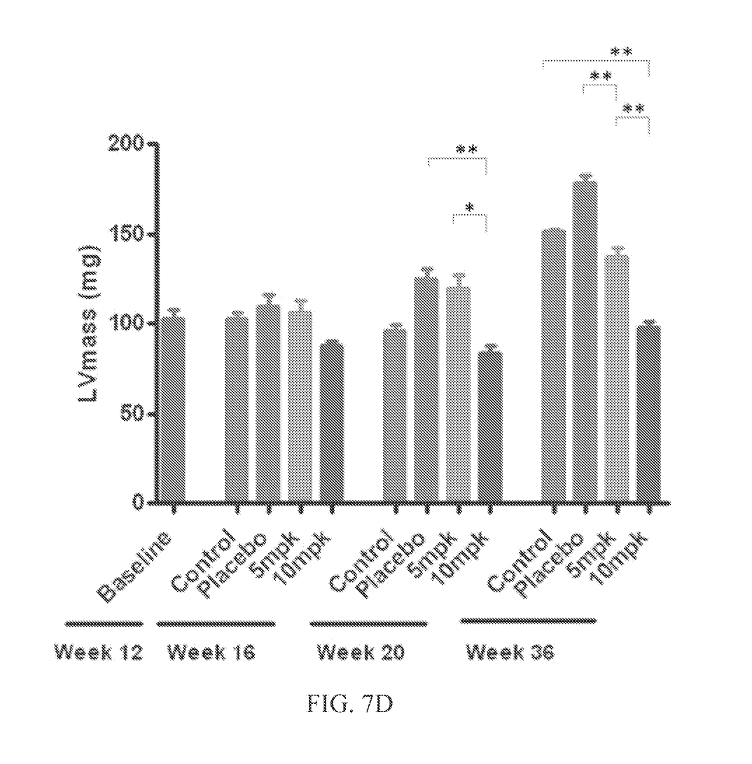

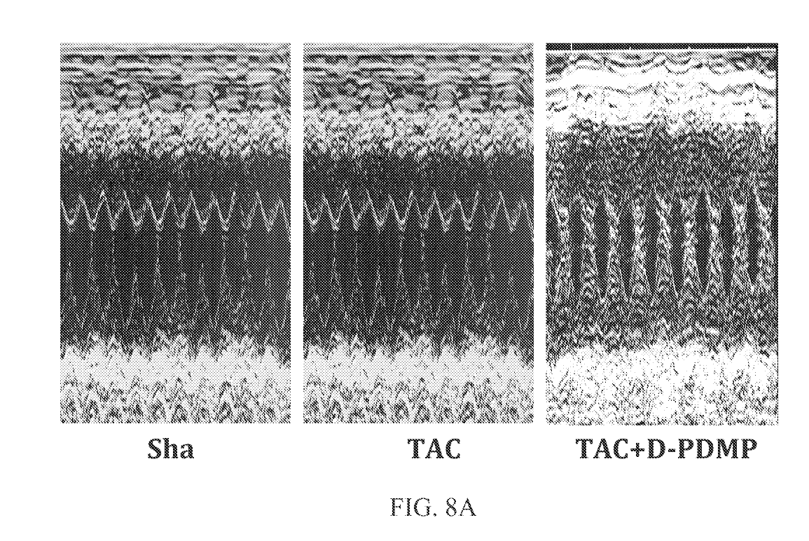

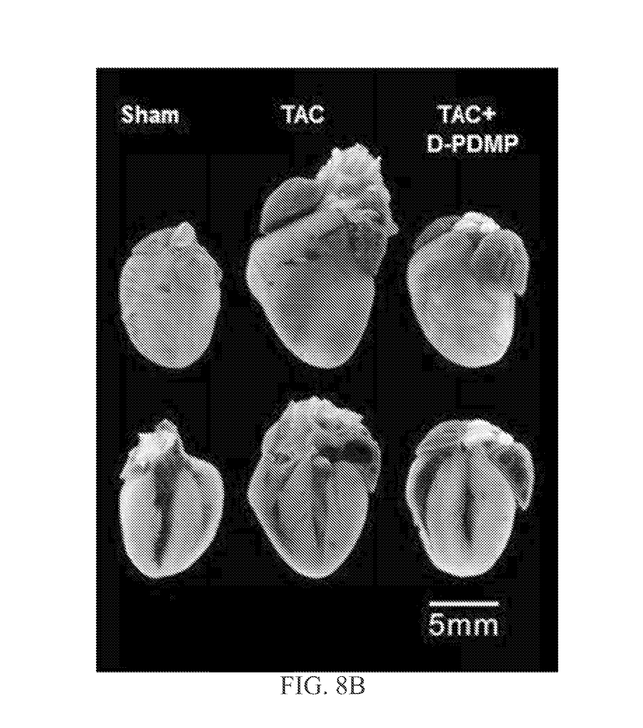

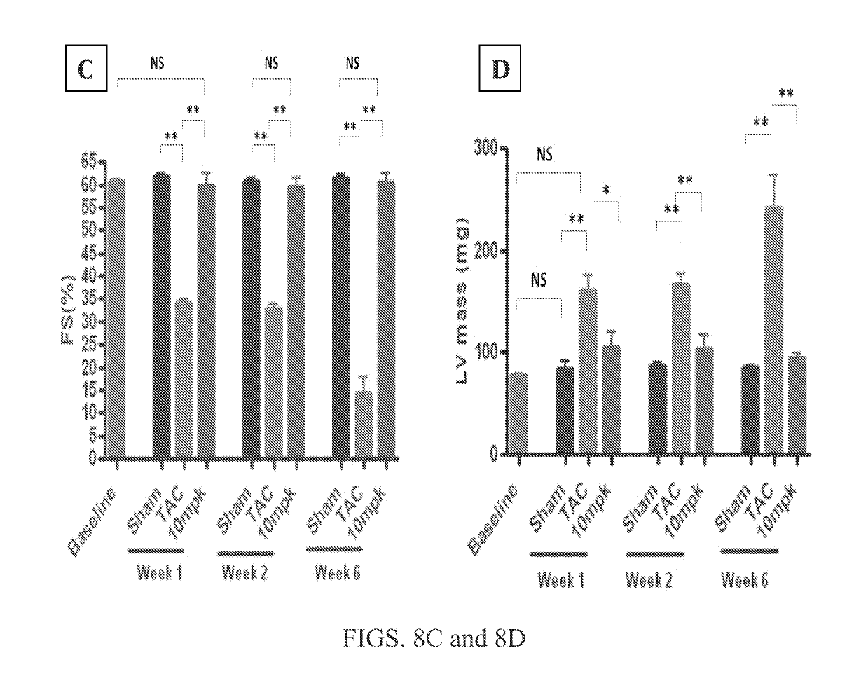

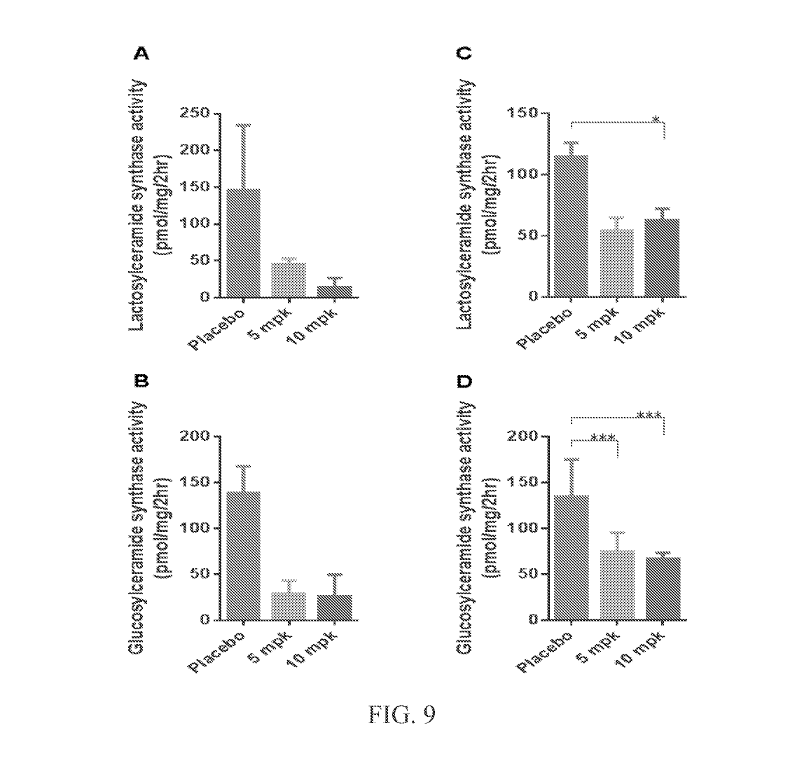

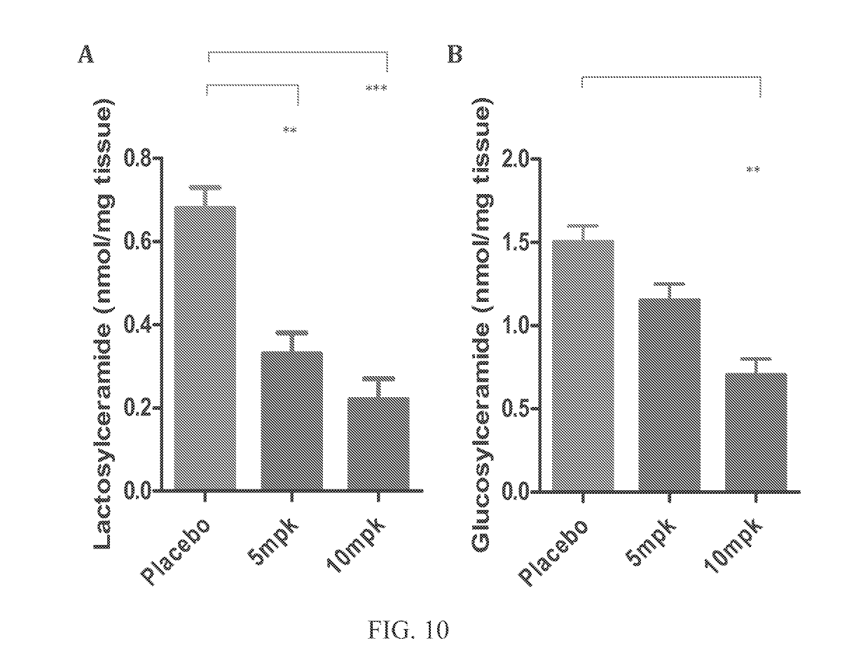

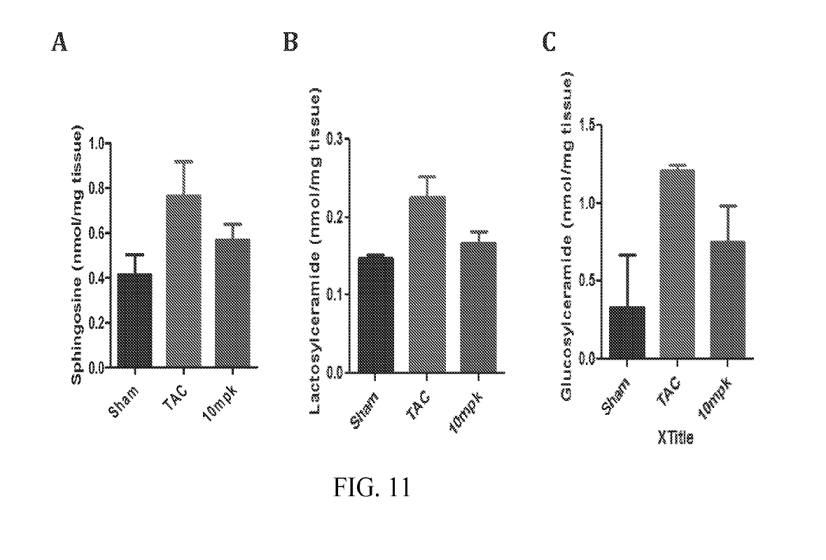

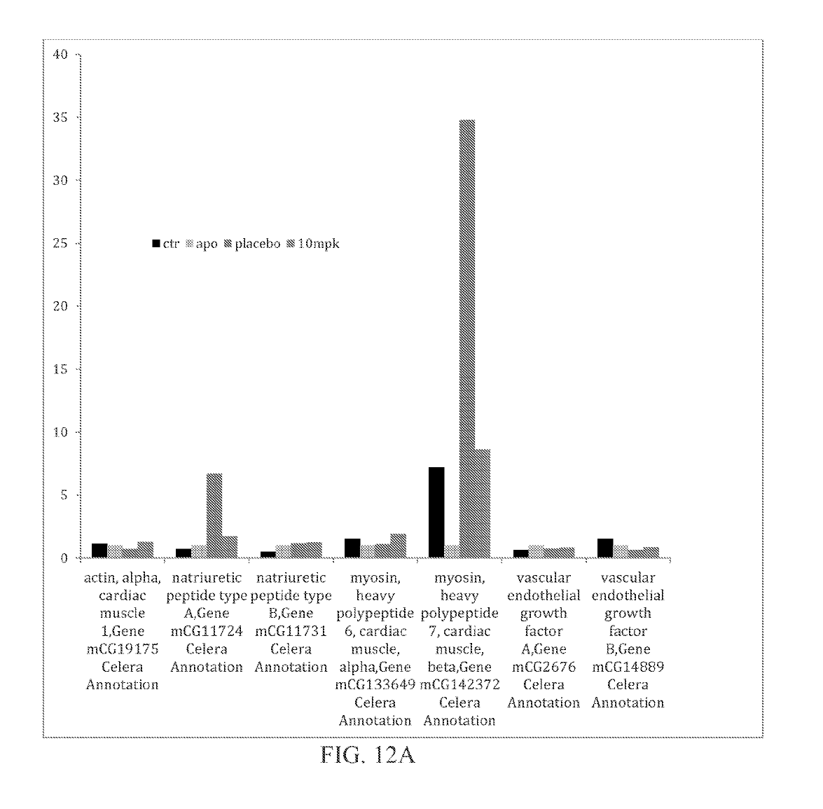

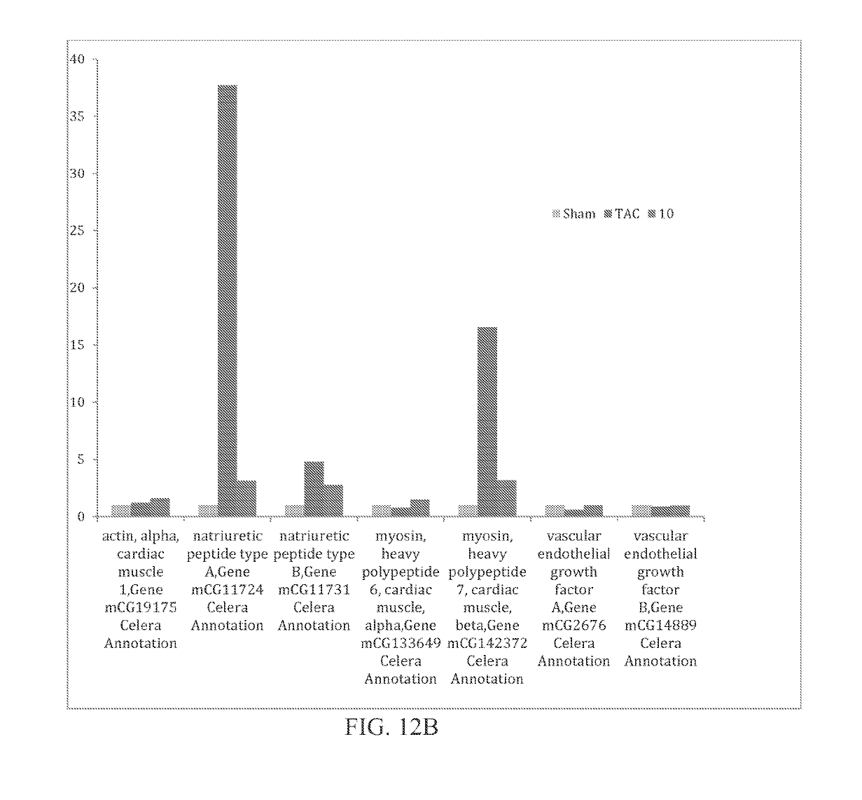

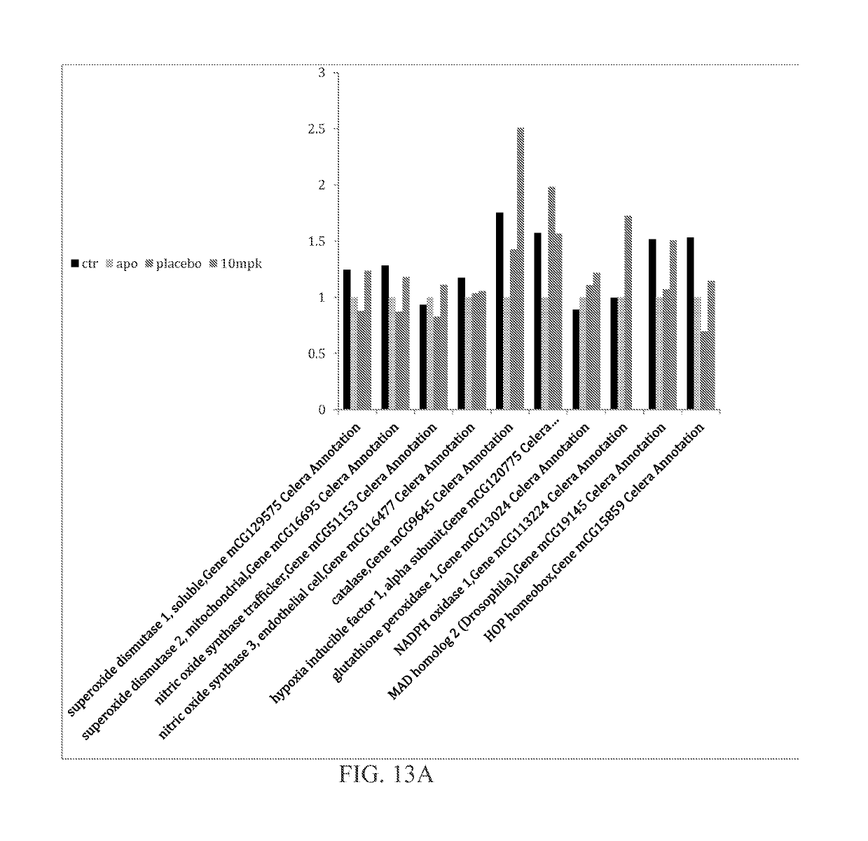

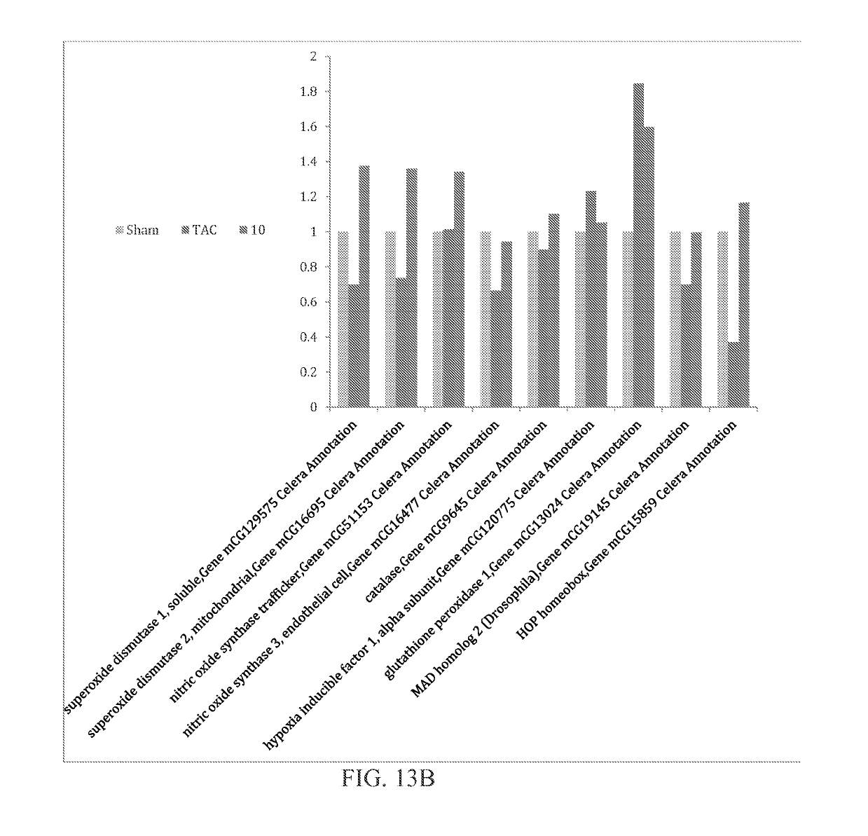

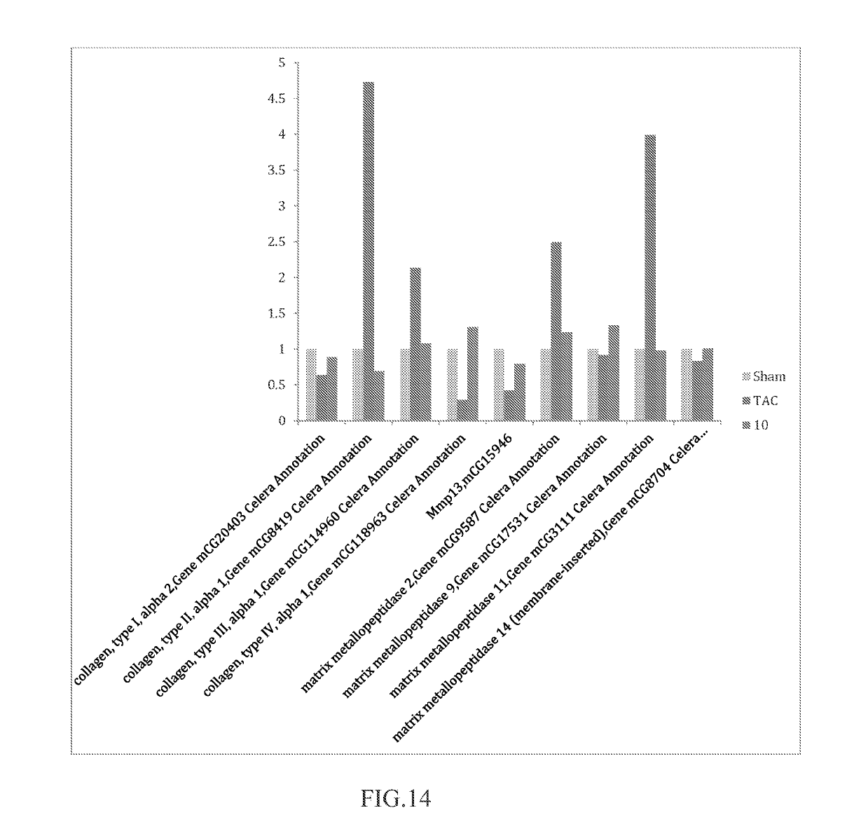

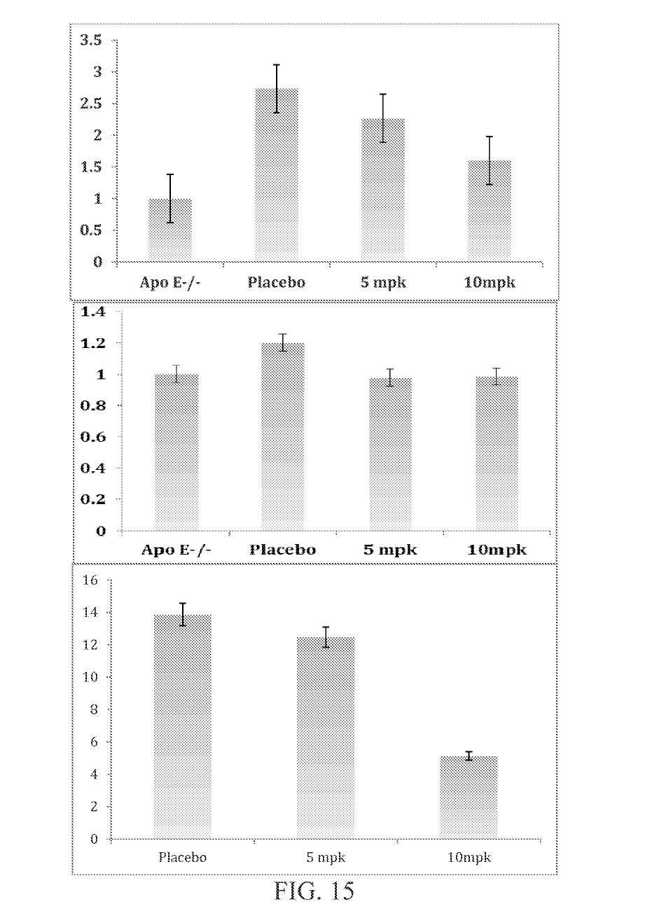

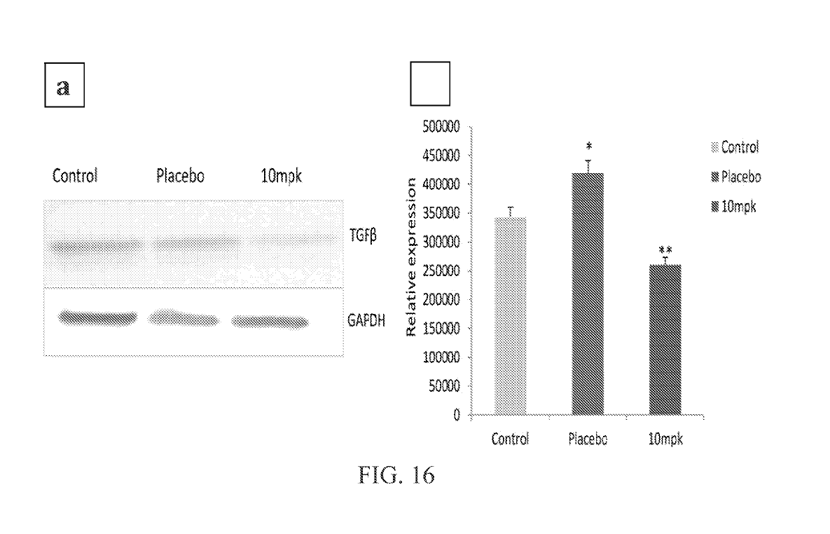

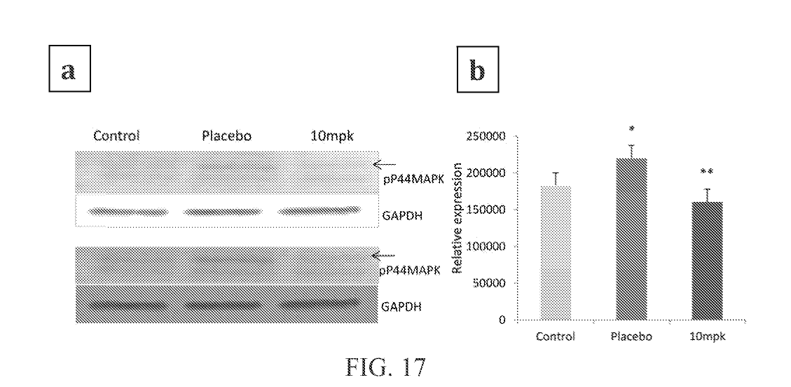

Cardiac hypertrophy involves the enlargement of the myocardial cells due to pressure and volume overload and contributes to the pathophysiology in various cardiovascular diseases. While several factors may contribute to cardiac hypertrophy, in our study we choose two experimental mouse models bring about this pathophysiolgy using different approaches. First we used the transgenic mouse model—the apolipoprotien-E−/− mouse that spontaneously develops atherosclerosis upon aging. However atherosclerosis in these mice can be expedited by feeding a western diet consisting of high fat and cholesterol. Marked hyperlipidemia and hyperlipoproteinemia raises blood viscosity and volume and thus contributes to cardiac hypertrophy. The second mouse model of cardiac hypertrophy in this study was the normal (C57 Bl-6) mouse subjected to trans-aortic constriction. See Jun et al., 209(2) ATHEROSCLEROSIS 381-86 (2010); Habashi et al., 120(18) CIRCULATION S963 (2009); and Takimoto et al., 11(2) NAT. MED. 214-22 (2005). This procedure raises blood volume and pressure contributing to cardiac hypertrophy, initially without hyperlipidemia and/or hyperlipoproteinemia. Nevertheless, we observed a time-dependent increase in cardiac hypertrophy in both these mouse models. This was exhibited by a continuous increase in the left ventricular mass and decreased fractional shortening thus reducing contractility (FIGS. 7, 8). Feeding mice D-PDMP in HCHF fed apoE−/− mice exerted a dose-dependent reversal in both the left ventricular mass and fractional shortening to base line levels. In the TAC mice, the extent of increase in left ventricular mass over a 6 week period of observation was increased several magnitude levels higher than the apoE−/− fed western diet. Feeding D-PDMP for a week before the surgical procedure completely reversed the left ventricular mass and fractional shortening to a similar level observed in sham mice heart. Previous studies have shown that structurally diverse agonists can induce the phosphorylation of p44 mitogen-activated protein kinase, implicated in cell proliferation. In agreement, we have observed that feeding a western diet to apoE−/− mice increases the serum level of oxidized -LDL about 4-fold and this was mitigated by treatment with D-PDMP. Additional studies have also shown that the phosphorylation of p44MAPK also accompanies cardiac hypertrophy and that it is required to activate the sodium hydrogen exchanger protein-1. Therefore, one mechanistic explanation for our observations is that because D-PDMP mitigated the phosphorylation of p44MAPK in heart tissue in these two diverse animal models, as a consequence it ameliorated cardiac hypertrophy. Transforming growth factor (TGF-B) levels have been shown to also increase in cardiac hypertrophy via the angiotensin-I type 2 receptor signaling and losartan can mitigate this phenotype also involving p44MAPK. In agreement, we observed a marked increase in the protein mass of TGF-B and increase in the expression of a host of other biomarker genes in both the mouse models of cardiac hypertrophy, and this was mitigated by the use of D-PDMP.

In sum, we report that inhibiting glycosphingolipid glycosyltransferase activity and glycosphingolipid production by the use of D-PDMP is a novel approach to mitigate cardiac hypertrophy in several mouse models. Thus drugs targeting the p44 MAPK and or upstream signaling intermediates leading to cardiac hypertrophy and or other heart diseases may be a novel therapeutic approach to prevent these diseases.

### Example 3

Determine the Long-Term (36 weeks) Effect of Treating the apoE−/− Mice with D-PDMP (5 and 10 mpk) on the Cardiovascular Parameters Above

### Example 4

Determine the Efficacy of D-PDMP in Interfering with Atherosclerosis and Cardiovascular Pathology Induced by Feeding a High Fat and Cholesterol Diet

### Example 5

Determine Whether Increase Activity/Mass/Gene Expression of Glycosyltransferase Is a Biomarker of Cardiac and Aortic Hypertrophy and Fibrosis Using RT-PCR and Other Molecular Assays

### Example 6

Determine the Effects of D-PDMP on Established Biomarkers of Cardiac Hypertrophy, Fibrosis, Proliferation Angiogenesis and Apoptosis.

1. A method for treating or preventing cardiac hypertrophy in a patient comprising the step of administering a therapeutically effective amount of a glycolipid synthesis inhibitor.

2. The method of claim 1, wherein the glycolipid synthesis inhibitor inhibits glucosylceramide synthase.

3. The method of claim 1, wherein the glycolipid synthesis inhibitor inhibits lactosylceramide synthase.

4. The method of claim 1, wherein the glycolipid synthesis inhibitor is D-threo-1-Phenyl-2-decanoylamino-3-morpholino-1-propanol HCl (D-PDMP).

5. The method of claim 1, wherein the glycolipid synthesis inhibitor is a D-PDMP analog.

6. The method of claim 5, wherein the D-PDMP analog is selected from the group consisting of D-threo-1-(3′,4′-ethyleneidoxy)phenyl-2-palmitoylaminod-3-pyrrolidino-1-propanol (EtDO-P4), D-threo-4′-hydroxy-1-phenyl-2-palmitoylamino-3-pyrrolidino-1-propanol (pOH-P4), D-threo-1-(3′,4′-trimethylenedioxy)phenyl-2-palmitoylamino-3-pyrrolidino-1-propanol (trimethylenedioxy-P4), D-threo-1-(3′,4′-methylenedioxy)phenyl-2-palmitoylamino-3-pyrrolidino-1-propanol (methylenedioxy-P4), and adamantan-1-yl glucosyl ceramide, (1R,2R) nonanoic acid[2-3-(2′,3′-dihydro-benzo[1,4]dioxin-6-′yl)-2-hydroxy-1-pyrrolidin-1-ylmethyl-ethyl]-1 amide-L-tartaric acid salt (Genz-1223346).

7. The method of claim 1, wherein the glycolipid synthesis inhibitor is 1,5-(butylimino)-1,5-dideoxy-D-glucitol (NB-DNJ).

8. The method of claim 7, wherein the glycolipid synthesis inhibitor is a NB-DNJ analog.

9. The method of claim 8, wherein the NB-DNJ analog is selected from the group consisting of N-nonyldeoxynojirimycin (NN-DNJ), N-butyl-deoxygalatconojirimycin (NB-DGJ), and N-(5-adamantane-1-yl-methoxy)pentyl)-deoxynojirimycin (AMP-DNJ or AMP-DNM).

10. The method of claim 1, wherein the glycolipid synthesis inhibitor is a small molecule, an antibody, a protein, a peptide, or a nucleic acid.

11. A method for treating or preventing cardiac hypertrophy in a patient comprising the step of administering an effective amount of a glucosylceramide synthase inhibitor.

12. A method for treating or preventing cardiac hypertrophy in a patient comprising the step of administering an effective amount of a lactosylceramide synthase inhibitor.

13. A method for treating or preventing cardiac hypertrophy and/or aortic intimal media thickening in a patient comprising the step of administering a therapeutically effective amount of a glycolipid synthesis inhibitor.

14. A method for treating or preventing aortic intimal media thickening in a patient comprising the step of administering a therapeutically effective amount of a glycolipid synthesis inhibitor.

15. A method for treating or preventing atherosclerosis in a patient comprising the step of administering a therapeutically effective amount of a glycolipid synthesis inhibitor.

16. A method for diagnosing cardiac hypertrophy in a patient comprising the steps of:
a. obtaining biological sample from the patient;
b. determining the level of expression of a glycosyltransferase biomarker in the sample; and
c. diagnosing the patient has having cardiac hypertrophy if the level of expression of the glycosyltransferase biomarker is significantly increased relative to a baseline level of the biomarker that is indicative of not having cardiac hypertrophy.

17. The method of claim 16, wherein the determining step is performed using mass spectrometry.

18. The method of claim 16, wherein the determining step is performed using an immunoassay.

19. A method for diagnosing cardiac hypertrophy in a patient comprising the steps of:
a. determining the level of expression of a glycosyltransferase biomarker in a sample obtained from the patient;
b. comparing the level of expression of the biomarker in the sample to reference levels of the biomarker that correlate to having cardiac hypertrophy and not having cardiac hypertrophy using a classification algorithm; and
c. diagnosing the patient has having cardiac hypertrophy if the level of expression of the glycosyltransferase biomarker correlates to a reference level of the biomarker that is indicative of not having cardiac hypertrophy.

20. The method of claim 19, wherein the determining step is performed using an immunoassay or mass spectrometry.

21. A method for treating cardiac hypertrophy in a patient comprising the steps of:
a. determining the level of expression of a glycosyltransferase biomarker in a sample obtained from the patient;
b. comparing the level of expression of the biomarker in the sample to reference levels of the biomarker that correlate to having cardiac hypertrophy and not having cardiac hypertrophy using a classification algorithm; and
c. treating the patient with an appropriate cardiac hypertrophy treatment if the level of expression of the glycosyltransferase biomarker correlates to a reference level of the biomarker that is indicative of not having cardiac hypertrophy.
